SEQUENCE LISTING (23 sequences)

SEQ ID NO: 1 - protein, 9 aa, Artificial Sequence
  Synthetic
EPRREVAEL

SEQ ID NO: 2 - protein, 9 aa, Artificial Sequence
  Synthetic
EPRRAVAEL

SEQ ID NO: 3 - protein, 9 aa, Artificial Sequence
  Synthetic
EPRRAVAAL

SEQ ID NO: 4 - protein, 9 aa, Artificial Sequence
  Synthetic
EPRREVAEL

SEQ ID NO: 5 - protein, 9 aa, Artificial Sequence
  Synthetic
EPRRAVAEL

SEQ ID NO: 6 - protein, 9 aa, Artificial Sequence
  Synthetic
EPRRAVAAL

SEQ ID NO: 7 - protein, 4 aa, Artificial Sequence
  Synthetic
AAAA

SEQ ID NO: 8 - protein, 20 aa, Artificial Sequence
  Synthetic
KIPKASSVPTELSAISTLYL

SEQ ID NO: 9 - protein, 15 aa, Artificial Sequence
  Synthetic
KLTWQELYQLKYKGI

SEQ ID NO: 10 - protein, 431 aa, Artificial Sequence
  Synthetic
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 11 - protein, 288 aa, Artificial Sequence
  Synthetic
MVGVGGGDVEDVTPRPGGCQISGRAARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 12 - protein, 33 aa, Artificial Sequence
  Synthetic
KIPKASSVPTELSAISTLYLAAAAEPRREVAEL

SEQ ID NO: 13 - protein, 33 aa, Artificial Sequence
  Synthetic
KIPKASSVPTELSAISTLYLAAAAEPRRAVAEL

SEQ ID NO: 14 - protein, 33 aa, Artificial Sequence
  Synthetic
KIPKASSVPTELSAISTLYLAAAAEPRRAVAAL

SEQ ID NO: 15 - protein, 33 aa, Artificial Sequence; identical to SEQ ID NO: 13
  Synthetic

SEQ ID NO: 16 - protein, 33 aa, Artificial Sequence
  Synthetic
KIPKASSVPTELSAIATLYLAAAAEPRRAVAAL

SEQ ID NO: 17 - protein, 33 aa, Artificial Sequence; identical to SEQ ID NO: 12
  Synthetic

SEQ ID NO: 18 - protein, 31 aa, Artificial Sequence
  Synthetic
KLTWQELYQLKYKGIGGGAAAAEPRREVAEL

SEQ ID NO: 19 - protein, 20 aa, Artificial Sequence
  Synthetic
KIPKACCVPTELSAISMLYL

SEQ ID NO: 20 - protein, 9 aa, Artificial Sequence
  Synthetic
EPRREVCEL

SEQ ID NO: 21 - protein, 20 aa, Artificial Sequence
  Synthetic
KIPKASSVPTELSAISTLYL

SEQ ID NO: 22 - protein, 15 aa, Artificial Sequence
  Synthetic
KLTWQELYQLKYKGI

SEQ ID NO: 23 - protein, 15 aa, Artificial Sequence
  Synthetic
KLTWQELYQLKYKGI

(19) United States
(12) Patent Application Publication
(10) Pub. No.: US 2014/0235542 A1
(43) Pub. Date: Aug. 21, 2014

(54) MODULAR, BIOACTIVE PEPTIDES FOR BINDING NATIVE BONE AND IMPROVING BONE GRAFT OSTEOINDUCTIVITY

(71) Applicant: WISCONSIN ALUMNI RESEARCH FOUNDATION, Madison, WI (US)

(72) Inventors: Sheeny K.L. Levengood, Madison, WI (US); Sabrina H. Brounts, Madison, WI (US); William L. Murphy, Waunakee, WI (US); Jae Sung Lee, Middleton, WI (US)

(73) Assignee: WISCONSIN ALUMNI RESEARCH FOUNDATION, Madison, WI (US)

(21) Appl. No.: 13/770,046

(22) Filed: Feb. 19, 2013

(51) Int. Cl. A61K 38/16

(52) U.S. Cl. CPC A61K 38/16
USPC 514/13.3; 514/16.7

(57) ABSTRACT

A modular peptide design strategy wherein the modular peptide has two functional units separated by a spacer portion is disclosed. More particularly, the design strategy combines a bone-binding portion and a biomolecule-derived portion. The modular peptides have improved non-covalent binding to the surface of native bone, and are capable of initiating osteogenesis, angiogenesis, and/or osteogenic differentiation.

### STATEMENT OF GOVERNMENT SUPPORT

This invention was made under AR052893 awarded by the National Institutes of Health. The government has certain rights in the invention.

### INCORPORATION OF SEQUENCE LISTING

A paper copy of the Sequence Listing and a computer readable form of the sequence listing containing the file named “28243-168 (P110341US01)_ST25.txt” which is 13,540 bytes in size (measured in MS-DOS) are provided herein and are herein incorporated by reference. This Sequence Listing consists of SEQ ID NOs:1-23.

### BACKGROUND OF THE DISCLOSURE

The present disclosure relates generally to modular biologically active molecules for binding native bone. Particularly preferred modular biologically active molecules may include modular cytokines, growth factors, hormones, nucleic acids, and fragments thereof Of particular importance in this disclosure are modular growth factors having improved non-covalent binding to native bone and being capable of initiating osteogenesis, angiogenesis, and/or osteogenic differentiation.

Natural proteins often contain at least two functional domains, which are linked together to form one multi-functional protein molecule. Specifically, these proteins are capable of activating cell surface receptors, and also binding with high affinity and specificity to natural extracellular matrices (ECMs). To achieve these diverse functions, a strategy commonly employed by nature involves creating modular proteins, in which distinct domains within a single protein are designed to enable either cell signaling or ECM binding. For example, the bone ECM protein osteocalcin (OCN) binds to hydroxyapatite (HA), the major mineral component in the ECM of bony tissues, with high affinity via an N-terminal domain, and also plays a critical role in regulating bone matrix formation via a C-terminal domain.

The mechanisms that enable the binding of signaling molecules to ECM in nature can potentially be extended to synthetic biomaterials as well. For example, a recent study indicated that it is possible to mimic nature's modular cell adhesion proteins (e.g. OCN, bone sialoprotein (BSP)) by engineering synthetic modular peptide molecules that bind to synthetic HA, yet remain capable of affecting cell adhesion. This modular design approach has been used to promote cell adhesion to natural and synthetic HA-based materials, which are now used in a wide range of common clinical orthopedic applications. However, previous studies have not been able to actively induce new bone formation by bone precursor cells, nor are they able to induce differentiation of stem cells into bone-forming cells.

Musculoskeletal conditions represent an average of 3% of the gross domestic product of developed countries, consuming an estimated $254 billion annually in the United States. Bone and joint diseases account for half of all chronic conditions in people over the age of 50, and the predicted doubling of this age group's population by 2020 suggests that the tremendous need for novel bone repair and replacement therapies will continue to grow rapidly. Emerging therapeutic approaches have focused on delivering growth factor molecules to skeletal defects, as these molecules are capable of actively inducing new bone formation. However, growth factor delivery strategies often result in sub-optimal delivery kinetics, and are difficult to incorporate into standard clinical procedures. These limitations complicate clinical translation of growth factor delivery in orthopedic applications.

Further, even though several synthetic bone graft substitutes have been developed, native bone grafts including autologous bone graft (autograft) and allogenic bone graft (allograft) still remain a popular choice in current clinical settings as they typically offer superior biological healing activity and structural strength when compared to synthetic counterparts.

Bone autograft is the gold standard for bone grafting transplantation and most commonly used to treat bone defects, as it is histocompatible and non-immunogenic as well as osteoconductive, osteoinductive and pro-osteogenic. However, its clinical use is often limited by the availability of autograft and potential donor site morbidity from bone graft harvesting. Thus, bone allograft has become an attractive alternative to avoid those problems as it is harvested from cadaver bone and readily available off-the-shelf with various shape and size. Allograft, although osteoconductive, generally lacks the ability to actively direct skeletal tissue repair to an extent depending on the preparation protocols. In response to this drawback, osteoinductive factors and osteogenic cells have been incorporated with bone allografts to accelerate and promote bone healing.

Accordingly, there is a need for modular growth factors that can be engineered to bind strongly to HA and HA-based materials, and particularly native bone, thereby forming a biologically active “molecular coating” with controllable characteristics. Specifically, it would be advantageous if the modular growth factor had two functional units, similar to natural proteins: a HA-binding sequence to allow for improved binding to the surfaces of HA and HA-based materials; and a biomolecule-derived sequence inspired by natural biologically active molecules such as bone morphogenetic protein-2 (BMP-2) and vascular endothelial growth factor (VEGF). These modular growth factors may be broadly applicable in orthopedics, as HA is among the most commonly used materials in orthopedic applications, including total joint replacements, trauma, and fracture healing.

### SUMMARY OF THE DISCLOSURE

Accordingly, the present disclosure is generally directed to modified peptides having improved non-covalent binding to the surfaces of a biomaterial, and in particular native bone. More specifically, in one aspect, the present disclosure is directed to a modular peptide for non-covalently binding to a surface of a HA-based biomaterial. The modular peptide comprises a hydroxyapatite-binding portion, a spacer portion, and a biomolecule-derived portion.

In some embodiments, the modular peptide is a modular growth factor such as BMP-2, BMP-7, fibroblast growth factor-2 (FGF-2), or vascular endothelial growth factor (VEGF). These modular growth factors are capable of both binding with high affinity and with spatial control to the surface of native bone and a “bone-like” HA-coated material and initiating at least one biological response such as osteogenesis, angiogenesis, or osteogenic differentiation.

In another aspect, the present disclosure is directed to a method of coating a biomaterial with a modular peptide. In one embodiment, the method comprises: exposing a biomaterial to a phosphate buffered saline (PBS) solution containing the modular peptide.

In some embodiments, the PBS solution includes from about 100 μg to about 1500 μg of a modular peptide. More particularly, in some embodiments the PBS solution includes from about 200 μg to about 750 μg of a modular peptide, and in some embodiments, the PBS solution includes about 500 μg of a modular peptide.

Furthermore, in some embodiments, the modular peptide is a modular growth factor such as BMP-2, BMP-7, FGF-2, and VEGF.

In another aspect, the present disclosure is directed to a method of coating native bone with a modular peptide. The method comprises: exposing native bone to a solution containing a modular peptide, wherein the modular peptide comprises a bone-binding portion and a biomolecule-derived portion and wherein the modular peptide is non-covalently bound to the native bone. Native bone may be, for example, a bone autograft, a bone allograft, and a bone xenograft.

In another aspect, the present disclosure is directed to a method of treating a bone fracture. The method comprises exposing fractured bone to a solution containing a modular peptide, wherein the modular peptide comprises a bone-binding portion and a biomolecule-derived portion; and incubating the bone and the modular peptide for a time sufficient to allow the modular peptide to non-covalently bind to the bone.

### BRIEF DESCRIPTION OF THE DRAWINGS

The disclosure will be better understood, and features, aspects and advantages other than those set forth above will become apparent when consideration is given to the following detailed description thereof. The application file contains at least one drawing executed in color. Copies of this patent or patent application publication with color drawing(s) will be provided by the Office upon request and payment of the necessary fee. Such detailed description makes reference to the following drawings, wherein:

FIG. 1A shows a SEM image (magnification of ×1000) of the HA-material layer grown on the PLG film in Example 1.

FIG. 1B shows a SEM image (magnification of ×1500) of the HA-material layer grown on the PLG film in Example 1.

FIG. 1C shows a SEM image (magnification of ×30000) of the HA-material layer grown on the PLG film in Example 1.

FIG. 1D shows a XRD spectrum of the HA-material layer grown on the PLG film in Example 1.

FIG. 2A shows the binding efficiency of the various modular peptides of Example 1 on the HA-coated PLG films.

FIG. 2B shows the binding isotherm of eBGa3 of Example 1 on the HA-coated PLG films as a function of peptide concentration.

FIG. 2C shows the release kinetics of eBGu1 and eBGu3 of Example 1 on the HA-coated PLG films.

FIG. 2D shows the release kinetics of eBGa1 and eBGa3 of Example 1 on the HA-coated PLG films.

FIG. 3A shows the effect of soluble modular peptides on ALP activity in hMSCs as measured in Example 1.

FIG. 3B shows the effect of soluble modular peptides on mineralized tissue formation by hMSCs as measured in Example 1.

FIG. 4A shows the effect of immobilized modular peptides on ALP activity in hMSCs as measured in Example 1.

FIG. 4B shows the effect of immobilized modular peptides on mineralized tissue formation by hMSCs as measured in Example 1.

FIG. 4C shows the effect of immobilized modular peptides on BMP-2 secretion by hMSCs as measured in Example 1.

FIG. 4D shows the effect of immobilized modular peptides on OCN production by hMSCs as measured in Example 1.

FIG. 5A shows the primers used in measuring the expression of osteogenic markers in Example 1.

FIG. 5B shows the effect of the immobilized modular peptides on expression of osteogenesis-related genes in hMSCs as measured in Example 1.

FIG. 5C shows the effect of the immobilized modular peptides on OCN expression by hMSCs over time as measured in Example 1.

FIG. 5D shows the effect of the immobilized modular peptides on OPN expression by hMSCs over time as measured in Example 1.

FIG. 5E shows the effect of the immobilized modular peptides on Cbfa1 expression by hMSCs over time as measured in Example 1.

FIG. 6 shows the binding isotherm of modular eBGa3 peptide to HA particles over time at 37° C. as measured in Example 1.

FIG. 7A shows the high performance liquid chromatography (HPLC) spectrum of modular VEGF-OCN peptide in Example 2.

FIG. 7B shows a MALDI-TOF spectrum of modular VEGF-OCN peptide in Example 2.

FIG. 7C shows circular dichroism (CD) spectrum of modular VEGF-OCN peptide in Example 2.

FIG. 8A shows the binding isotherm of modular VEGF-OCN peptide on HA particles as measured in Example 2. Empty symbols represent VEGF-OCN and filled symbol represents VEGF-mimic.

FIG. 8B shows the binding isotherm of modular VEGF-OCN peptide on HA particles over time as measured in Example 2.

FIG. 8C shows fluorescently labeled VEGF-OCN peptide bound on HA particles.

FIG. 8D shows qualitative comparison of the binding of VEGF-OCN (top) and VEGF-mimic (bottom) on HA particles.

FIG. 9A shows optical micrographs showing the effect of soluble modular peptides on C166-GFP cell proliferation as determined in Example 2.

FIG. 9B shows the effect of soluble modular peptides on C166-GFP cell proliferation in Example 2.

FIG. 10A shows fluorescence micrographs of C166-GFP cells cultured on VEGF-OCN or VEGF-mimic immobilized on HA slab in Example 2.

FIG. 10B shows the effect of immobilized modular peptides on C166-GFP cell proliferation in Example 2.

FIG. 11A shows a fluorescence micrograph of eBGa3 peptides that are incorporated on a HA slab using dip coating.

FIG. 11B shows a fluorescence micrograph of eBGa3 peptides that are incorporated on a HA slab using stamping.

FIG. 11C shows a fluorescence micrograph of eBGa3 peptides that are incorporated on a HA slab using a painting method.

FIG. 11D shows a fluorescence micrograph of VEGF-OCN peptides that are incorporated on a HA slab using a painting method.

FIG. 11E shows a fluorescence micrograph of VEGF-OCN peptides that are incorporated on a HA slab using a painting method.

FIG. 12 shows fluorescence images of cortical bone samples after incubation in rhodamine-labeled mBMP solutions with different concentrations for different time periods as evaluated in Example 3. Green and red fluorescence were emitted from native bone (autofluorescence) and rhodamine, respectively.

FIG. 13A shows the quantification of fluorescence intensity of rhodamine-labeled mBMP bound to cortical bone by incubating in various conditions as analyzed in Example 3. Data are shown as mean±standard deviation. *p<0.01 and **p<0.05.

FIG. 13B shows the quantification of fluorescence intensity of rhodamine-labeled mBMP and mBMP-mut bound to cortical bone by incubating in 100 μg/mL peptide solution for different time periods as analyzed in Example 3. Data are shown as mean±standard deviation. *p<0.01 and **p<0.05.

FIG. 14 shows P values from Student's t-test between the amount of modular peptide bound to cortical bone by incubating in peptide solution with various concentrations as analyzed in Example 3.

FIG. 15A shows fluorescence images of trabecular bone cores after incubating in rhodamine-labeled mBMP solution for different time periods in a bone bioreactor as analyzed in Example 3. Data are shown as mean±standard deviation. **p<0.05.

FIG. 15B shows fluorescence intensity of trabecular bone cores after incubating in rhodamine-labeled mBMP solution for different time periods in a bone bioreactor as analyzed in Example 3. Data are shown as mean±standard deviation. **p<0.05.

FIG. 16 shows P values from Student's t-test between the amount of modular peptide bound to cortical bone by incubating in peptide solution for various time periods as analyzed in Example 3.

FIG. 17 shows fluorescence images of (A) cortical bone; (B) trabecular bone dip-coated in rhodamine-labeled mBMP solution; (C) cortical bone spotted with rhodamine-labeled mBMP solution and (D) “UW” written with rhodamine-labeled mBMP solution on cortical bone as analyzed in Example 3.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

The present disclosure is generally directed to a modular peptide design, and more particularly, to a modular peptide design that includes both a modular growth factor-derived portion to induce stem cell differentiation, and further, a binding portion that allows for improved binding of the modular peptides to native-bone and “bone-like” HA-based or HA-coated biomaterials. The approach was designed to promote differentiation of human mesenchymal stem cells (hMSCs) into osteoblasts. MSCs are capable of differentiating into multiple cell lineages, including osteoblasts, chondrocytes and adipocytes.

As used herein, “biomolecule-derived portion” and “modular growth factor-derived portion” (used above) of the modular peptide are used interchangeably herein to refer to the portion of the modular peptide that is capable of stimulating cellular growth, proliferation and differentiation. The “biomolecule-derived portion” may be all of or a portion of a polypeptide known by one skilled in the art to contain the functional domain of the original protein. For example, where the “biomolecule-derived portion” is obtained from a growth factor, the “biomolecule-derived portion” may be part of the growth factor that functions to stimulate the cell. The terms “bone-binding portion” or “HA-binding portion” are used herein to refer to the portion of the modular peptide that non-covalently binds to the native bone or the “bone-like” HA-based or HA-coated biomaterials.

As described herein, the modular peptide may be considered a fusion (or chimeric) protein in which the biomolecule-derived portion is joined to the bone-binding (or HA-binding) portion to form a single polypeptide with functional properties derived from each of the portions. Thus, one portion of the modular peptide may be capable of stimulating cellular growth, proliferation and differentiation (i.e., the biomolecule-derived portion) and the other portion of the modular peptide may be capable of non-covalently binding native bone or “bone-like” HA-based or HA-coated biomaterials (i.e., the bone-binding (or HA-binding) portion).

Osteoblast differentiation has been shown to be regulated by multiple proteins, including bone morphogenetic proteins (BMPs) and Wnt. Among them, BMP-2 is one of the most potent inducers of osteogenic MSC differentiation in vitro and in vivo. BMP-2 promotes osteogenic differentiation by up-regulation expression of bone-related proteins, including osteocalcin (OCN), osteopontin (OPN), and alkaline phosphatase (ALP).

Based on the multifunctional properties of natural skeletal proteins (e.g., osteocalcin) and the inductive effects of BMP-2 on hMSC differentiation, a modular peptide design strategy that has two functional units has been developed. More particularly, in one embodiment, the design strategy combines a HA mineral binding portion (also referred to herein as hydroxyapatite-binding portion) and a biomolecule-derived portion. It was further found that binding to HA-based biomaterials, and subsequent release, could be varied significantly by changing the sequence of the hydroxyapatite-binding portion. It has further been unexpectedly found that, in addition to binding “bone-like” HA-based or HA-coated biomaterials, the HA binding portion also non-covalently binds to native bone. Thus, the HA mineral binding portion of the modular peptides is also referred to as “a bone-binding portion.” The bone-binding portion (or HA-binding portion) of the modular peptide includes a peptide sequence that allows for the non-covalent binding of the bone-binding portion of the modular peptide to native bone or “bone-like” HA-based coated biomaterials.

In one embodiment, the first unit of the modular peptide includes a peptide sequence inspired by an N-terminal α-helix in the protein osteocalcin (OCN), which is known to bind strongly to the crystal lattice of HA-mineral. Hydoxyapatite (HA) is a major mineral component of vertebrate bone tissue and has been widely used in orthopedic applications since the early 1980s due to its favorable interactions with native bone tissue, which is often termed “bioactivity.” Specifically, HA has been used clinically as a bone void filler, a non-load-bearing implant (e.g., for nasal septal bone and middle ear), and as a coating on metallic implants to promote their fixation to bone and limit the need for cemented fixation. In each case, the goal of these devices is to promote bone growth upon or within an implant, and HA encourages the process by promoting proliferation and matrix synthesis by bone-forming cells.

Preferably, the first hydroxyapatite-binding portion (also referred to herein as bone-binding portion) (e.g., SEQ ID NO:1) includes a peptide sequence inspired by the 5.7 kDa native protein osteocalcin (OCN), and more specifically, by the 9-mer sequence on the N-terminus of OCN. Osteocalcin-HA binding is largely mediated via the peptide sequence of OCN, which contains three γ-carboxylated glutamic acid (Gla) residues at positions 1, 5, and 8 that coordinate with calcium ions in the HA crystal lattice to promote high levels of binding.

Alternatively, it has been found that at least one or all three Gla residues present in SEQ ID NO:1 can be substituted with either glutamic acid (Glu) or alanine (Ala). Specifically, in some embodiments, the peptide sequences of SEQ ID NO:2 (γ-carboxylated glutamic acid (Gla) residues at positions 1 and 8 and Ala residue at position 5); SEQ ID NO:3 (γ-carboxylated glutamic acid (Gla) residue at position 1 and Ala residues at positions 5 and 8); SEQ ID NO:4 (Glu residues at positions 1, 5, and 8); SEQ ID NO:5 (Glu residues at positions 1 and 8 and Ala residue at position 5); and SEQ ID NO:6 (Glu residue at position 1 and Ala residues at positions 5 and 8) may be used as the hydroxyapatite-binding portion (see Table 1). The Glu and Ala substitutions can influence the charge density and secondary structure of the peptide molecules, and therefore, influence the peptide-HA binding.

TABLE 1
Sequences of Glu and Ala substituted hydroxyapatite-binding portion
of OCN 9-mer.

| SEQ | | |
|---|---|---|
| ID NO | Peptide | Amino Acid Sequence |
| 1 | γ-carboxylated glutamic acid (Gla) residues at | γEPRRγEVAγEL |
| | positions 1, 5, and 8 | |
| 2 | γ-carboxylated glutamic acid (Gla) residues at | γEPRRAVAγEL |
| | positions 1 and 8 and Ala residue at position 5 | |
| 3 | γ-carboxylated glutamic acid (Gla) residues at | γEPRRAVAAL |
| | position 1 and Ala residues at positions 5 and 8 | |
| 4 | Glu residues at positions 1, 5, and 8 | EPRREVAEL |
| 5 | Glu residues at positions 1 and 8 and Ala residue | EPRRAVAEL |
| | at position 5 | |
| 6 | Glu residue at position 1 and Ala residues at | EPRRAVAAL |
| | positions 5 and 8 | |

In another aspect, the present disclosure is directed to a method of coating native bone with a modular peptide. Typically, supraphysiological concentrations of osteoinductive proteins are combined with a variety of artificial bone grafts using a carrier material that releases the proteins at the defect site. The binding of the modular peptide to native bone provides the advantage of being used in lower doses than typically used with carrier materials. This allows for minimal side effects and maximal bone regeneration.

The modular peptide further includes a second unit that is a biomolecule-derived portion capable of initiating osteogenesis, angiogenesis, and/or osteogenic differentiation. For example, in one preferred embodiment, the second unit is a biomolecule-mimic portion derived from the 20-mer “knuckle” epitope of BMP-2 protein (SEQ ID NO:8), disclosed in U.S. Pat. No. 7,132,506 to Kyocera Corporation (Nov. 7, 2006). Specifically, it has been previously found that various forms of BMP-2 are capable of enhancing bone formation at ectopic and orthotopic sites, including recombinant BMP-2 protein delivered exogenously and BMP-2 protein synthesized in vivo upon expression of BMP-2 encoding DNA. BMP-2 has also become an important component of emerging stem cell-based tissue regeneration approaches, as stem cell fate decisions are often regulated by growth factor signaling. For example, BMP-2 has been shown to promote differentiation of human mesenchymal stem cells down the osteogenic lineage in standard pro-osteogenic cell culture conditions.

Another suitable growth factor includes the 15-mer sequence derived from VEGF (SEQ ID NO:9). Other suitable growth factors that can be used in the biomolecule-derived portion (i.e., second unit) of the modular peptide include sequences derived from BMP-7 (SEQ ID NO:10) and FGF-2 (SEQ ID NO:11).

To control the spacing between the HA-binding (bone-binding) portion and the biomolecule-derived portion, a spacer portion is present in the modular peptide. It is believed that the bioactivity of the biomolecule-derived portion in the modular peptide may be increased with an increase in the spacer length. More particularly, it is hypothesized that too little of a spacing between the surface of the biomaterial and the biomolecule-derived portion may not be optimal for the biomolecule-derived portion's bioactivity as the biomolecule-derived portion may be too close to the biomaterial surface to be accessible to cell receptors. Accordingly, by controlling the spacing between the HA-binding (bone-binding) portion and the biomolecule-derived portion, the level of bioactivity by the biomolecule-derived portion can be controlled. Generally, the spacer portion can be any amino acid sequence capable of forming an α-helix with the HA-binding (bone-binding) portion. For example, in one or more embodiments, the spacer portion may be an alanine (Ala)ₙ spacer, such as the (Ala)₄ spacer having the sequence of SEQ ID NO:7. This spacer portion is particularly preferred for use as it is capable of being both a spacer and an extension, as the HA-binding (bone-binding) portion and poly (Ala) sequences have a known propensity to form α-helices. Other suitable spacer portions may include a leucine (Leu)ₙ spacer, a lysine (Lys)ₙ spacer, and a glutamate (Glu)ₙspacer.

Other suitable spacer portions may include a polyethylene glycol spacer such as 3500 Da polyethylene glycol and 5000 Da polyethylene glycol.

The modular peptides of the present disclosure may be synthesized by standard solid-phase synthesis, such as by using Fmoc-protected amino acids and purified by HPLC. For example, in one embodiment, the modular peptides are synthesized by solid-phase peptide synthesis on Fmoc-Rink Amide MBHA resin with Fmoc-protected a-amino groups via peptide synthesizer (CS Bio, Menlo Park, Calif.). The side-chain-protecting groups used can be: t-butyl for Tyr, Thr and Ser; 2,2,5,7,8-pentamethyl-chroman-6-sulfonyl for Arg; t-BOC for Lys; and t-butyl ester for Gla and Glu. In some cases, 5(6)-FAM (5(6)-carboxyfluorescein, Sigma) is conjugated to the N-terminal lysine residue to characterize binding and release kinetics of modular growth factors on HA-coated biomaterials. The resulting peptide molecules can be cleaved from resin for 4 hours using a TFA:TIS:water (95:2.5:2.5) cocktail solution, filtered to remove resin, and precipitated in diethyl ether. Crude peptide mixtures can be purified using a Shimadzu Analytical Reverse Phase-HPLC (Vydac C18 column) with 1%/min of 0.1% TFA in acetonitrile (ACN) for 60 minutes.

It should be understood by one skilled in the art that various other known methods for preparing modular peptides can also be used without departing from the scope of the present disclosure. For example, in one alternative embodiment, the modular peptides are synthesized manually with PyBop/DIPEA/HOBT activation.

Suitable modular peptides of the present disclosure include those having a sequence selected from SEQ ID NO:12, SEQ ID NO:13, SEQ ID NO:14, SEQ ID NO:15, SEQ ID NO:16, SEQ ID NO:17, and SEQ ID NO:18.

The present disclosure is further directed to methods of coating biomaterials with the modular peptides described above. Generally, a biomaterial, such as hydroxyapatite or hydroxyapatite-based materials, is coated by exposing the biomaterial to a solution including the modular peptide. In one embodiment, the biomaterial is exposed to the solution using a dip coating method. Other suitable methods for exposing the biomaterial to a solution including the modular peptide include spotting, stamping, brushing, direct writing, and painting.

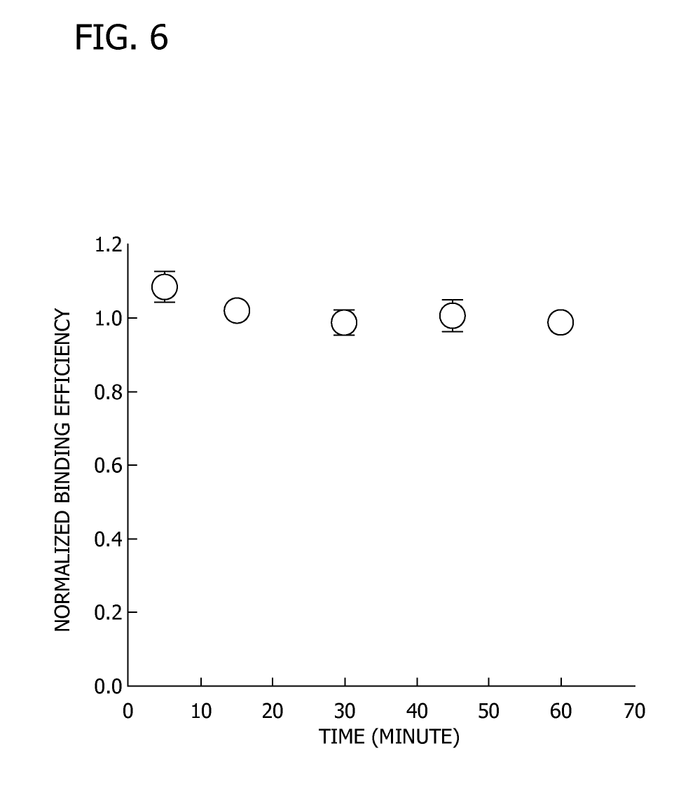

For example, in one or more embodiments, a hydroxyapatite-based material is exposed to a phosphate buffered solution (PBS) including from about 200 mg to about 750 mg of a modular peptide. More particularly, the PBS solution included from about 200 mg to about 750 mg of a modular peptide having a sequence selected from SEQ ID NO:12 (γ-carboxylated glutamic acid (Gla) residues at positions 25, 29, and 32), SEQ ID NO:13 (γ-carboxylated glutamic acid (Gla) residues at positions 25 and 32 and Ala residue at position 29), SEQ ID NO:14 (γ-carboxylated glutamic acid (Gla) residue at position 25 and Ala residues at positions 29 and 32), SEQ ID NO:15 (Glu residues at positions 25 and 32 and Ala residue at position 29), SEQ ID NO:16 (Glu residue at position 25 and Ala residues at positions 29 and 32), SEQ ID NO:17 (Glu residues at positions 25, 29 and 32), or SEQ ID NO:18 (Glu residues at positions 23, 27, and 30). In one particular embodiment, HA particles were exposed to SEQ ID NO:12 in 10 μM PBS peptide solution (pH 7.4) for a period of 60 minutes. The amount of peptide bound on the HA particles was normalized by the mean of all values, and the results are shown in FIG. 6.

It should be noted that although discussed herein using a PBS solution, any carrier solution known in the art for including a modular peptide can be used in the methods of the present disclosure. For example, other suitable solutions include HEPES buffer solution, PIPES buffer solution, Tris buffer solution, saline solution, and the like.

Typically, the biomaterial is exposed to the solution including the modular peptide under constant agitation.

In another embodiment, the method of coating native bone with a modular peptide includes exposing native bone to a solution having the modular peptide. Suitable solutions may be, for example, phosphate buffered saline (PBS), HEPES buffer solution, PIPES buffer solution, Tris buffer solution, saline solution, and the like.

The native bone may be exposed to the solution having the modular peptide by placing the native bone into the solution and incubating the native bone in the solution for a suitable period of time to allow the modular peptide to non-covalently bind to the native bone. The native bone may be exposed to the solution having the modular peptide with or without agitation. The native bone may be exposed to the modular peptide in solution for a period of about 2 minutes to about 10 hours, and including about 30 minutes. Additionally, the native bone may be exposed to the solution having the modular peptide by dip coating, painting, stamping, spotting, brushing and combinations thereof

Suitable native bone may be, for example, a bone autograft, a bone allograft, and a bone xenograft. The term “bone autograft” is used herein according to its ordinary meaning as understood by those skilled in the art to refer to bone that is obtained from a subject who serves as both the donor and recipient of the native bone. The term “bone allograft” is used herein according to its ordinary meaning as understood by those skilled in the art to refer to bone that is donated by a subject who is different than the recipient. The term “bone xenograft” is used herein according to its ordinary meaning as understood by those skilled in the art to refer to bone that is donated by one species that is different than the species of the recipient.

The concentration of the modular peptide in the solution may be from about 200 μg/mL to about 750 μg/mL. Particularly suitable concentrations of the modular peptide may be from about 50 μg/mL to about 150 μg/mL, including about 100 μg/mL.

In another aspect, the present disclosure is directed to a method of treating a bone fracture. The method includes exposing a bone having a fracture to a solution containing a modular peptide, wherein the modular peptide has a bone-binding portion and a biomolecule-derived portion; and incubating the bone and the modular peptide for a time sufficient to allow the modular peptide to non-covalently bind the bone.

The fractured bone may be, for example, complete fractures in which bone fragments separate completely, incomplete fractures in which bone fragments are partially joined, compression fractures, impacted fractures, avulsion fractures, stress fractures, capillary fractures, fissure fractures, greenstick fractures, insufficiency fractures, open fractures, closed fractures, pathologic fractures, spiral fractures, shear fractures, sprain fractures, comminuted fractures, and any combination thereof.

Suitable modular peptides may be those described herein.

The bone having the fracture may be exposed to a solution having the modular peptide by covering the exposed bone fracture with the solution. For example, an incision may be made to expose the bone fracture and the solution having the modular peptide may be poured over or pipetted onto the exposed bone. Alternatively, a syringe may be used to inject the solution having the modular peptide at the site of the bone fracture. Upon exposure, the modular peptide non-covalently binds to the bone in the region of the fracture where it may function in the healing of the bone fracture.

The disclosure will be more fully understood upon consideration of the following non-limiting Examples.

### EXAMPLE 1

In this Example, modular peptides were synthesized and used to coat a HA-based biomaterial. The binding efficiency and subsequent release of the modular peptides from the biomaterial was then analyzed. Additionally, the bioactivity of the biomolecule-derived portion used in the modular peptide was analyzed.

### Synthesis and Purification of Modular Growth Factors

To begin, multiple modular peptides (Table 2) were synthesized by solid-phase peptide synthesis on Fmoc-Rink Amide MBHA resin with Fmoc-protected α-amino groups via peptide synthesizer (CS Bio, Menlo Park, Calif.). The side-chain-protecting groups used were: t-butyl for Tyr, Thr and Ser; 2,2,5,7,8-pentamethyl-chroman-6-sulfonyl for Arg; t-BOC for Lys; and t-butyl ester for Gla and Glu. In some cases, 5(6)-FAM (5(6)-carboxyfluorescein, Sigma) was conjugated to the N-terminal lysine residue to characterize binding and release kinetics of modular growth factors on HA-coated polylactide-co-glycolide (PLG) films. The resulting peptide molecules were cleaved from resin for 4 hr using a TFA:TIS:water (95:2.5:2.5) cocktail solution, filtered to remove resin, and precipitated in diethyl ether. Crude peptide mixtures were purified using a Shimadzu Analytical Reverse Phase-HPLC (Vydac C18 column) with 1%/min of 0.1% TFA in acetonitrile (ACN) for 60 minutes and analyzed by MALDI-TOF mass spectrometry (Bruker Reflex II time-of-flight mass spectrometer).

TABLE 2
Sequences of modular peptide growth factors and
natural template

| Peptide | Amino Acid Sequence |
|---|---|
| Human BMP-2 | KIPKACCVPTELSAISMLYL (AAs: 73-92) |
| | (SEQ ID NO: 19) |
| Human OCN | γEPRRγEVCγEL (AAs: 17-25) (SEQ ID |
| | NO: 20) |
| eBMP2^{[a]} | KIPKASSVPTELSAISTLYL (SEQ ID NO: 21) |
| eBGa3^{[b]} | KIPKASSVPTELSAISTLYLAAAAγEPRRγEVAγEL |
| | (SEQ ID NO: 12) |
| eBGa2 | KIPKASSVPTELSAISTLYLAAAAγEPRRAVAγEL |
| | (SEQ ID NO: 13) |
| EBGa1 | KIPKASSVPTELSAISTLYLAAAAγEPRRAVAAL |
| | (SEQ ID NO: 14) |
| EBGu1 | KIPKASSVPTELSAIATLYLAAAAEPRRAVAAL |
| | (SEQ ID NO: 16) |
| eBGu3 | KIPKASSVPTELSAISTLYLAAAAEPRREVAEL |
| | (SEQ ID NO: 17) |

^{[a]}The eBMP2 peptide sequence was originally synthesized by Tanihara and co-workers. Cys and Met from human BMP-2 sequence were replaced by Ser and Thr.
^{[b]}Cys from human OCN sequence was replaced by Ala in modular peptides to avoid complicating disulfide linkages.

### PLG Film Preparation and Mineral Growth

Poly (lactide-coglycolide) (PLG) films were prepared via a solvent casting process in which PLG (85:15) pellets were dissolved in chloroform (50 mg/ml), added to a PTFE dish, and dried for 2 days. The films were further dried at 50-55° C. for 4 hr to remove residual solvent and samples were cooled to room temperature. Square films (1 cm²) were manually cut out of the resulting PLG film sheets. A “bone-like” HA-based material layer was grown on the PLG films using a direct deposition technique by biomimetic mineralization in modified simulated body fluid (mSBF).

The surface morphologies of HA-coated and uncoated PLG films were examined by scanning electron microscopy (SEM). A conductive gold coating was applied to the surface of each film via sputter coating, and samples were imaged under high vacuum using a LEO 1530 SEM (Zeiss, Oberkochen, Germany) operating at 10-30 kV. X-ray diffraction spectra of HA-coated and non-coated PLG films were collected using a Bruker Hi-Star 2-D X-ray diffractometer (XRD).

### Binding Isotherms and Release Kinetics of Modular Peptides

To measure the binding efficiency of modular peptides to the HA-coated PLG films and to gain preliminary insight into the properties that influence modular peptide immobilization, 1 cm² HA-coated PLG films was first exposed to PBS solutions containing 500 μg (1 mg/ml) of 5(6) FAM-conjugated eBMP-2, eBGu1, eBGu3, eBGa1, or eBGa3 modular peptide solution (See Table 1 for definitions of these abbreviations). The films were incubated in peptide solutions with constant agitation for 4 hr at 37° C., and the amount of free peptide remaining was determined by measuring the fluorescence emission of the solution (excitation: 494 nm; emission 519 nm) using a Synergy HT Multi-Detection Microplate Reader (BioTek, Winooski, Vt.), and comparing this emission to standard samples with known concentrations of 5(6)-FAM. To further characterize surface immobilization of the peptide with the highest binding efficiency—the eBGa3 peptide-1 cm² HA-coated films were incubated in various concentrations (50-750 nM) of 5(6)-FAM-conjugated eBGa3 peptide for 4 hr with constant agitation at 37° C. The amount of peptide bound to HA-coated film was again determined by fluorescence analysis, as described above.

To quantify release kinetics of 5(6) FAM-conjugated modular peptides from HA-coated film, the films were first incubated in solutions containing 250 μM (˜500 μg) of each peptide (eBGu1, eBGu3, eBGa1, or eBGa3) to allow for binding (as described above), then incubated in 500 μl of PBS buffer at 37° C. with constant agitation for 5 days (eBGu1 and eBGu3 peptides) or 10 weeks (eBGa1 and eBGa3 peptides), respectively. Whole buffer solutions were changed at indicated time points and the amount of peptide released from the HA-coated film was determined via fluorescence analysis and comparison with standards containing known amounts of 5(6)-FAM. The fluorescent images of fluorescently-labeled peptides bound to HA-coated films were obtained using an Olympus IX51 fluorescence microscope (Olympus, Center Valley, Pa.).

Culture of Human Mesenchymal Stem Cells (hMSCs)

hMSCs (Cambrex, Walkersville, Md., passages 5-6) were cultured in mesenchymal stem cell growth medium (MSCGM: Cambrex) consisting of MSC Basal Medium supplemented with 10% fetal bovine serum, L-glutamine, 100 units/ml penicillin, and 0.1 mg/ml streptomycin and grown using culture methods described elsewhere. 2.5×10⁴ hMSCs were seeded onto either tissue culture-treated polystyrene (TCP) or four different types of experimental substrates (1 cm²) (PLG, HA-coated PLG, eBGu3-treated HA coating, or eBGa3-treated HA coating). hMSCs were allowed to attach to each substrate overnight, then cultured in MSCGM with osteogenic culture supplements (OS) (0.1 μM dexamethasone, 50 μg/ml ascorbic acid, and 10 mM β-glycerophosphate) for 24 days. The effects of soluble peptides included in culture medium were evaluated by adding 50 μg of eBGu3 or eBGa3 peptides to hMSC cultures on TCP in 500 μl of medium with or without osteogenic culture supplements. In each experimental and control sample, whole volume medium changes were performed every 4 days by replacement with fresh medium and collected medium was used for BMP-2 and OCN ELISA assays.

### Quantification of Alkaline Phosphatase (ALP) Activity

The biological activity of modular peptides was initially assayed by their ability to enhance ALP activity in hMSCs. AP Assay Reagent S (GenHunter, Nashville, Tenn.) was used for cell staining and the EnzoLyte pNPP Alkaline Phosphatase Assay Kit (Anaspec, San Jose, Calif.) was used to measure enzymatic activity of ALP at day 12. For ALP staining, cells were washed with 1 ml of 1× PBS and 10% formalin, incubated at room temperature for 30 minutes, and washed again with PBS, and this wash was repeated 3 times. Cell layers were then stained with 0.5 ml of AP Assay Reagent S and incubated at room temperature for 30 minutes. Cell layers were washed 3 times with 1× PBS after staining was completed. Images of stained samples were captured via an Olympus IX-51 inverted microscope. For the ALP activity assay, cells were washed twice with a lysis buffer containing 0.1% Triton X-100. The lysate was centrifuged, and the resulting supernatant was assayed for ALP activity by incubating with 50 μl p-nitrophenyl phosphate (pNPP) in an assay buffer at 37° C. for 15 minutes. ALP activity was measured at 405 nm, and calculated as the ratio of p-nitrophenol released to total DNA concentration (nmol/min/ng DNA). To determine the amount of total DNA in each well, the cell nuclei were disrupted by addition of the aforementioned lysis buffer followed by centrifugation, and quantified using the CyQUANT Assay Kit (Molecular Probes, Eugene, Oreg.).

### Quantification of Alkaline Phosphatase (ALP) Activity

Characterization of mineralized tissue growth was performed via Alizarin Red-S (ARS) staining at day 20. The cultured cells on each type of biomaterial were washed with PBS and fixed in 10% (v/v) formaldehyde at room temperature for 30 minutes. The cells were then washed twice with excess distilled H₂O prior to addition of 1 ml of 40 mM ARS (pH 4.1) per well for 30 minutes. After aspiration of the unincorporated ARS, the wells were washed four times with 4 ml distilled H₂O while shaking for 10 minutes. For quantification of staining, 400 μl 10% (v/v) acetic acid was added to each well for 30 minutes with shaking. The cell monolayers were then scraped from the substrates and transferred with 10% (v/v) acetic acid to a 1.5-ml tube. After vortexing for 30 seconds, the slurry was overlaid with 250 μl mineral oil, heated to 85° C. for 10 minutes, and transferred to ice for 5 minutes. The slurry was then centrifuged at 15,000 g for 15 minutes and 300 μl of the supernatant was removed to a new 1.5-ml tube. Then, 200 μl of 10% (v/v) ammonium hydroxide was added to neutralize the acid. Aliquots (100 μl) of the supernatant were read in triplicate at 405 nm in 96-well plate reader.

### BMP-2 and Osteocalcin ELISAs

Two ELISA kits were used to quantify the secreted amount of BMP-2 (Quantikine BMP-2 Immunoassay, R&D Systems, Minneapolis, Minn.) and osteocalcin (Gla-type Osteocalcin EIA Kit, Zymed, Carlsbad, Calif.) in culture media according to manufacturer's instructions. Cell culture media were collected from various culture conditions at days 8, 16, and 24 and then measured for BMP-2 and osteocalcin protein levels.

### RNA Purification and RT-PCR Analysis

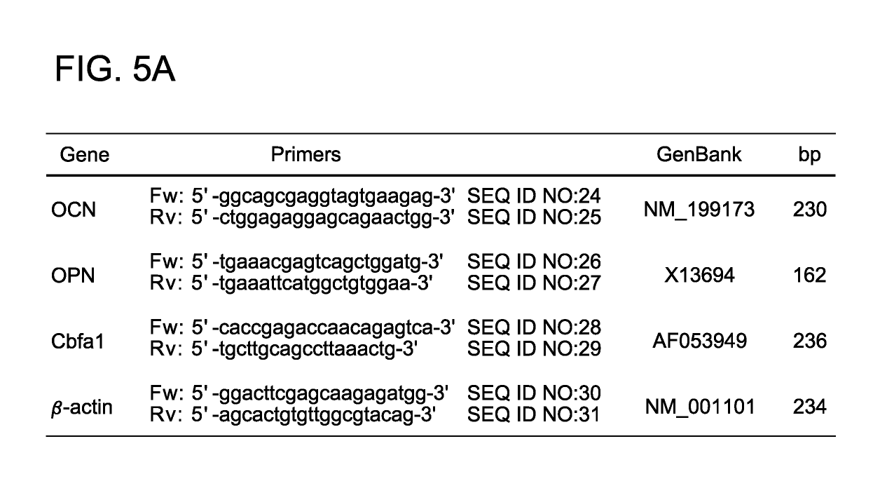

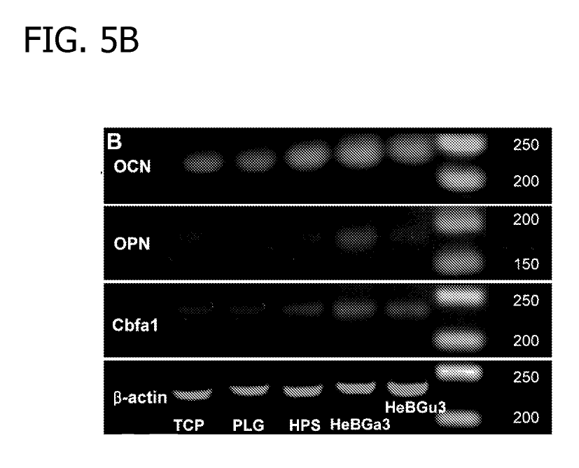

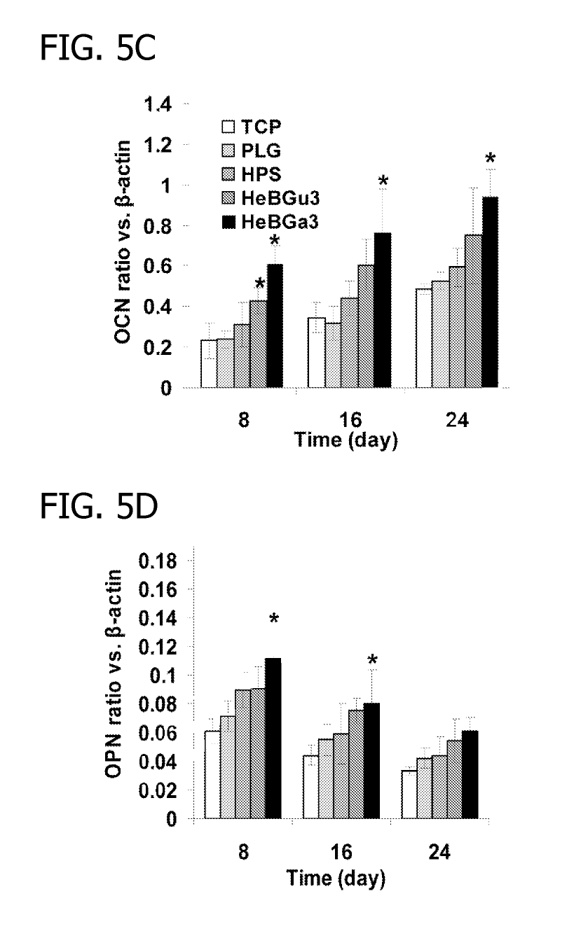

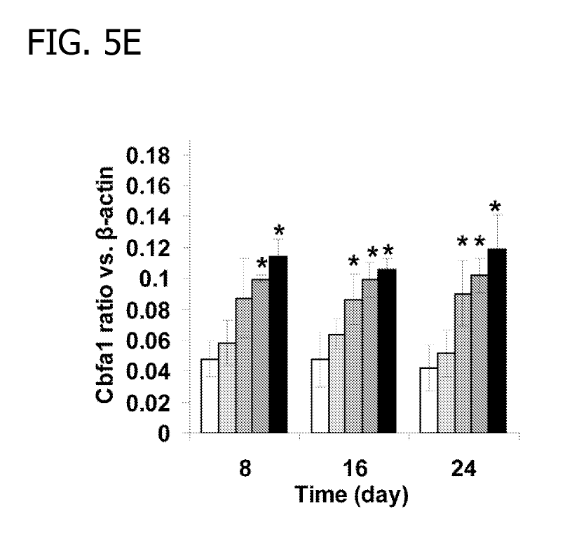

For mRNA analysis, the adherent cells were removed from culture dishes or each cultured substrate via 0.05% trypsin and resuspended in 350 μl RLT buffer (Qiagen, Valencia, Calif.). Total RNA was extracted using RNeasy mini-kits (Qiagen). First-strand cDNA was synthesized from 0.5 μg total RNA with 0.5 μg pd(T)12-18 as the first strand primer, using Ready-to-Go RTPCR Beads (GE Healthcare, Piscataway, N.J.), and then amplified by PCR using primer sets (FIG. 5A) in a Robocycler Gradient 96 (Stratagene, La Jolla, Calif.). Cycling conditions were as follows: 97° C. for 5 minutes followed by 32 cycles of amplification (95° C. denaturation for 30 seconds, 60° C. annealing for 30 seconds, 72° C. elongation for 30 seconds), with a final extension at 72° C. for 5 minutes. The PCR products were analyzed by electrophoresis on a 1.5% agarose gel stained with SYBR gold nucleic acid gel stain and relative gene ratios of OCN, OPN, and Cbfa1 versus-actin gene were measured by densitometry.

### Statistical Analysis

All data are given as mean±standard deviation. Statistical comparisons of the results were made using one way analysis of variance (ANOVA) with Dunnett's post hoc tests. Shapiro-Wilk method was used if a normality test was needed. The data analyses were performed with Statistical Program for the Social Sciences (SPSS) software and differences were considered significant at p<0.05 between control and experimental groups.

### Results

### Modular Peptide Binding and Release Kinetics

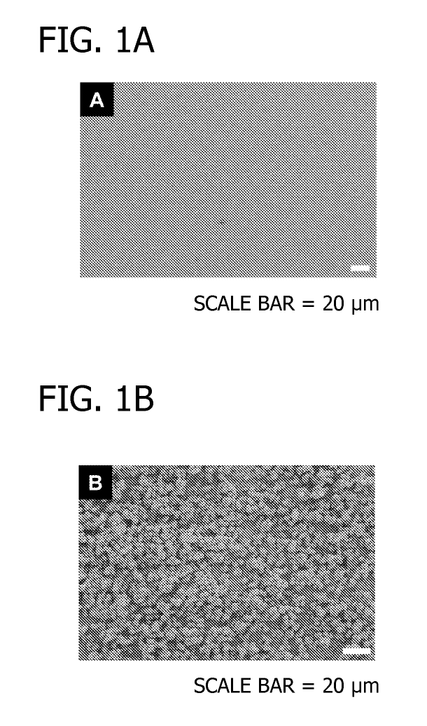

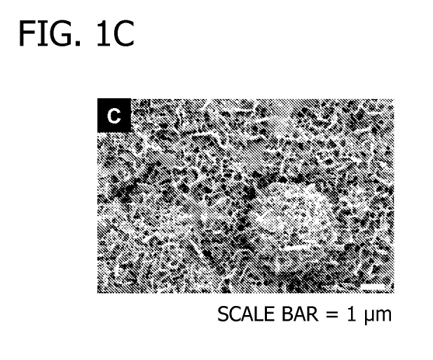

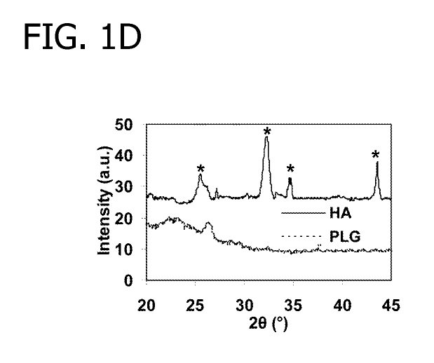

Specifically, SEM images (FIG. 1A-C) and XRD spectra FIG. 1D) demonstrated that the HA-mineral layer grown on the PLG film surface had a plate-like nanostructure and a HA phase, similar to vertebrate bone mineral in structure and composition.

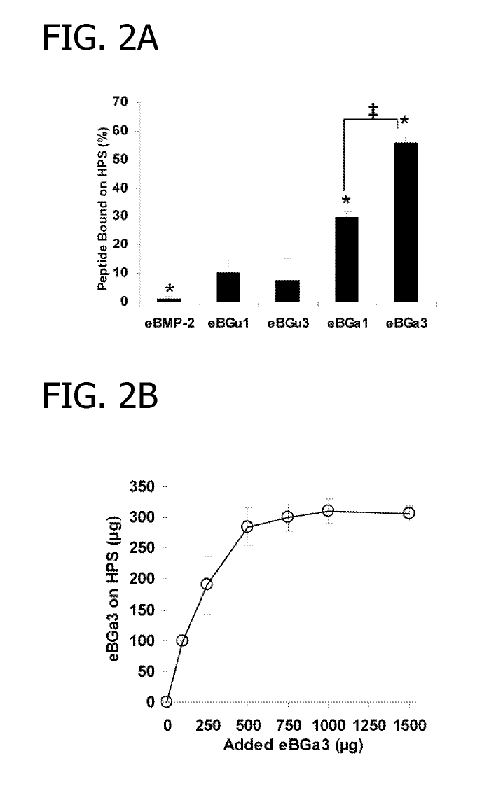

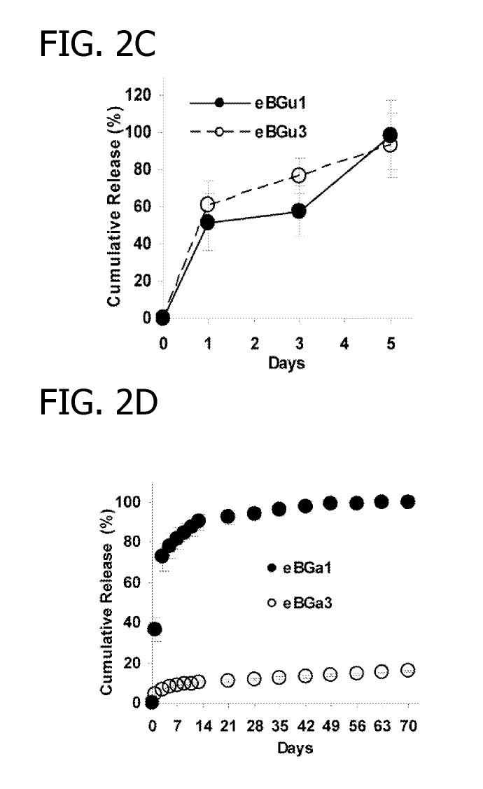

The binding efficiency of modular peptides on the HA-coated PLG films was sequence-dependent and increased in the following order: eBGu3 (7.6±7.8%)<eBGu1 (10.3±4.7%)<eBGa1 (29.9±2%)<eBGa3 (55.9±2.2%) (FIG. 2A). The binding efficiency of eBGa3 was substantially higher than other peptides studied (p*‡<0.005), and the binding of this molecule was thus studied in further detail. The amount of bound eBGa3 on the HA-coated film increased with peptide concentration and reached saturation at approximately 150 μM (300 μg) (FIG. 2B). The release kinetics of the modular peptides from HA-coated films were also highly dependent on the HA-binding portion (FIGS. 2C and D). eBGu1 (98.89±18.84% after 5 days) and eBGu3 (93.33±17.24% after 5 days) peptides were released rapidly from HA-coated films. In contrast, the eBGa3 peptide was released much more slowly, as only 15.7±0.6% of peptide was released after 70 days (FIG. 2D). Notably, these data indicate that nearly 85% of the initially bound eBGa3 peptide remained bound after 70 days.

### Biological Activity of Modular Peptides

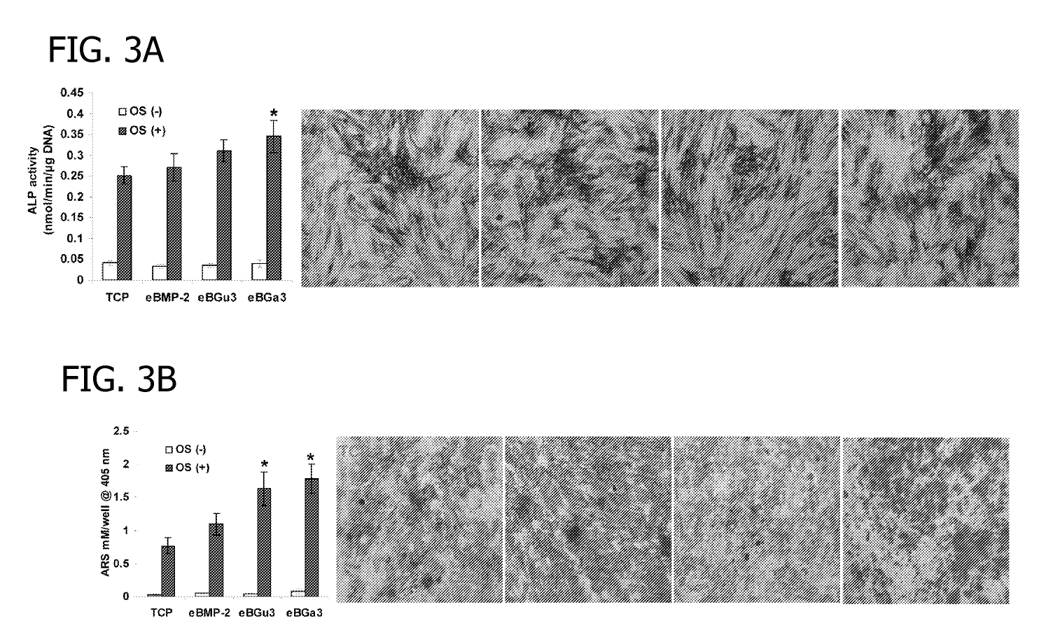

Soluble modular peptides added to hMSC growth medium along with osteogenic supplements had a positive influence on osteogenic differentiation of hMSCs. Specifically, the eBGa3 peptide significantly increased ALP activity (p=0.017) (FIG. 3A) and mineralized tissue formation (p=0.018) (FIG. 3B). Importantly, there were no significant differences between the positive effects of eBGu3 and eBGa3 when added as soluble supplements to standard hMSC culture, suggesting that the biological activity of the BMP2-derived portion of the peptides was not significantly influenced by the sequence of the HA-binding portion.

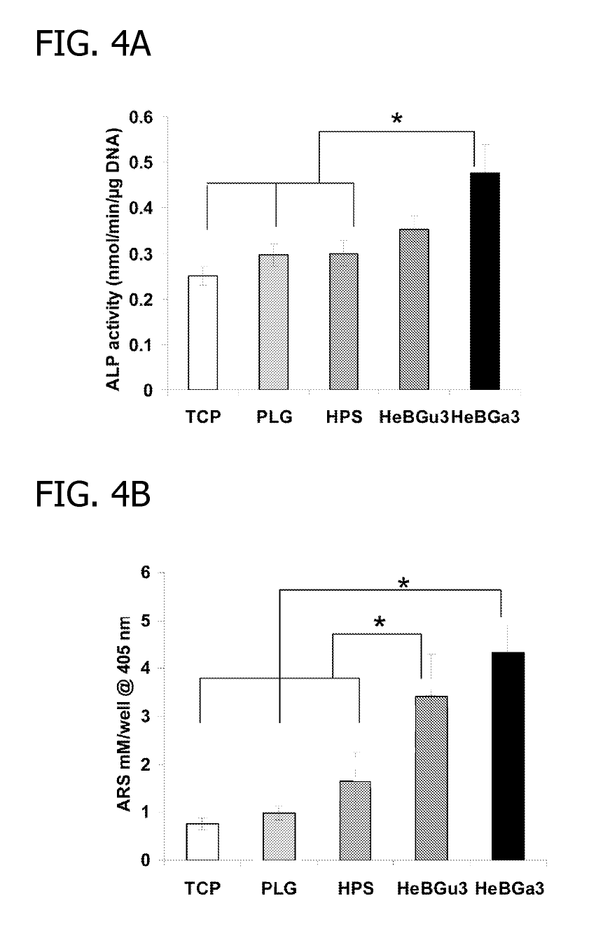

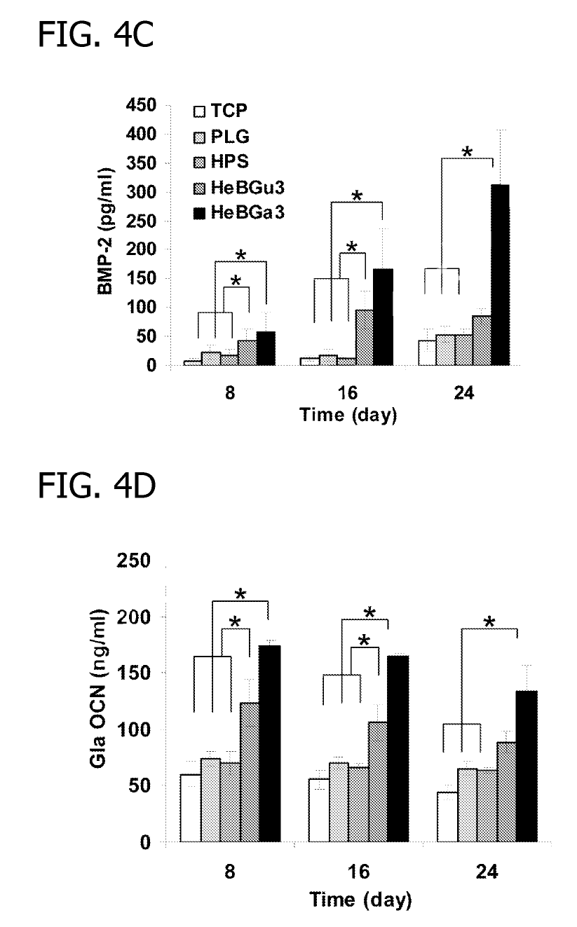

When bound to a HA-coated film, the eBGa3 peptide significantly enhanced ALP activity and mineralized tissue formation by hMSCs (FIGS. 4A and B). hMSCs cultured on eBGa3-bound, HA-coated films (termed “HeBGa3 substrates”) expressed significantly higher ALP activity (0.48±0.06 nmol/min/μg DNA) than hMSCs on untreated TCP (0.25±0.02), PLG (0.30±0.02), or HA-coated (0.30±0.03) films (FIG. 4A). Similarly, Alizarin red S staining of mineralized tissue was significantly increased on HeBGa3 substrates (4.32±0.57 mM/well) when compared to untreated TCP (0.76±0.12), PLG (0.98±0.14), or HA-coated (1.66±0.6) substrates (FIG. 4B). Importantly, HeBGa3 film substrates also induced enhanced BMP-2 secretion (FIG. 4C, days 16 and 24) and OCN production (FIG. 4D, days 8, 16, and 24) when compared to untreated substrates. Specifically, the hMSCs cultured on HeBGa3 produced a 6-fold higher amount of BMP-2 protein (311.59±94.55 pg/ml) when compared to TCP (43.36±18.60 pg/ml) at day 24 (p=0.002) (FIG. 4C), and OCN production was approximately 3-fold higher on HeBGa3 substrates (172.98±5.7 ng/ml) when compared to TCP substrates (60.21±10.62 ng/ml) on day 8 (p<0.0001) (FIG. 4D). Taken together, these data indicate that the eBGa3-treated substrates promote osteogenic differentiation of hMSCs.

The effects of eBGu3-treated, HA-coated films (termed “HeBGu3 substrates”) on osteogenic differentiation of hMSCs were less pronounced than the effects of the HeBGa3 substrates. Specifically, HeBGu3 substrates did not significantly enhance ALP activity of hMSCs (FIG. 4A), but did significantly enhance mineralized tissue formation (p<0.02) (FIG. 4B). Effects of HeBGu3 substrates on production of BMP2 and OCN were significant at day 8 and day 16, but not significant at day 24. These data indicate that the eBGu3-treated substrates can promote osteogenic differentiation of hMSCs, but the effects are not as substantial as the effects of eBGa3-treated substrates.

### Expression of Osteogenic Markers

Furthermore, the correlation of osteogenic differentiation to the expression levels of osteogenesis-related proteins, including OCN, osteopontin (OPN), and core-binding factor alpha 1 (Cbfa1) via RT-PCR using the primers indicated (FIG. 5A) were analyzed. OCN expression was significantly increased on HeBGa3 substrates at all time points studied when compared to TCP (p<0.01), PLG (p<0.01), and HPS (p<0.04) (FIGS. 5B and C). OPN expression was significantly increased on HeBGa3 substrates at day 8 (p=0.005) and day 16 (p=0.032) when compared to TCP (FIGS. 5B and D). Cbfa1 expression was increased on HeBGa3 substrates at all time points studied when compared to TCP (p<0.002) (FIGS. 5B and E). Expression of osteogenesis-related genes was also enhanced on HeBGu3 substrates compared to TCP, PLG, and HPS, but to a lesser extent than HeBGa3 substrates. Specifically, HeBGu3 substrates enhanced OCN expression at day 8 and enhanced Cbfa1 expression at all time points studied. Taken together, the RT-PCR analyses indicate that eBGa3-treated films promote expression of osteogenic markers to a greater extent than eBGu3-treated films, and this result is in agreement with the aforementioned analyses of ALP activity, mineralized tissue formation, BMP-2 production, and OCN production. It is noteworthy that Cbfa1 expression was also enhanced on HA-coated films when compared to TCP at day 16 (p=0.031) and day 24 (p<0.044), indicating that the HA-coated film alone slightly influences expression of pro-osteogenic transcription factors.

### EXAMPLE 2

In this Example, modular peptides were synthesized and used to coat a HA-based biomaterial. The binding behavior and bioactivity of the modular peptides was then analyzed.

Specifically, modular peptides (Table 3) were synthesized and analyzed using the methods described in Example 1.

TABLE 3
Sequences of modular peptide growth factors and natural
template

| Peptide | Amino Acid Sequence |
|---|---|
| Human OCN | γEPRRγEVCγEL (AAs: 17-25) (SEQ ID NO: 20) |
| VEGF helical region | KVKFMDVYQRSYCHP (AAs: 14-28) (SEQ ID NO: 22) |
| VEGF mimic* | KLTWQELYQLKYKGI (SEQ ID NO: 23) |
| VEGF-OCN | KLTWQELYQLKYKGI-GGGAAAA-γEPRRγEVAγEL |
| | (SEQ ID NO: 18) |

*First synthesized by Pedon, et al., inspired by the VEGF helical region (AAs: 14-25), PNAS, 102(4): 14215-14220 (2005).

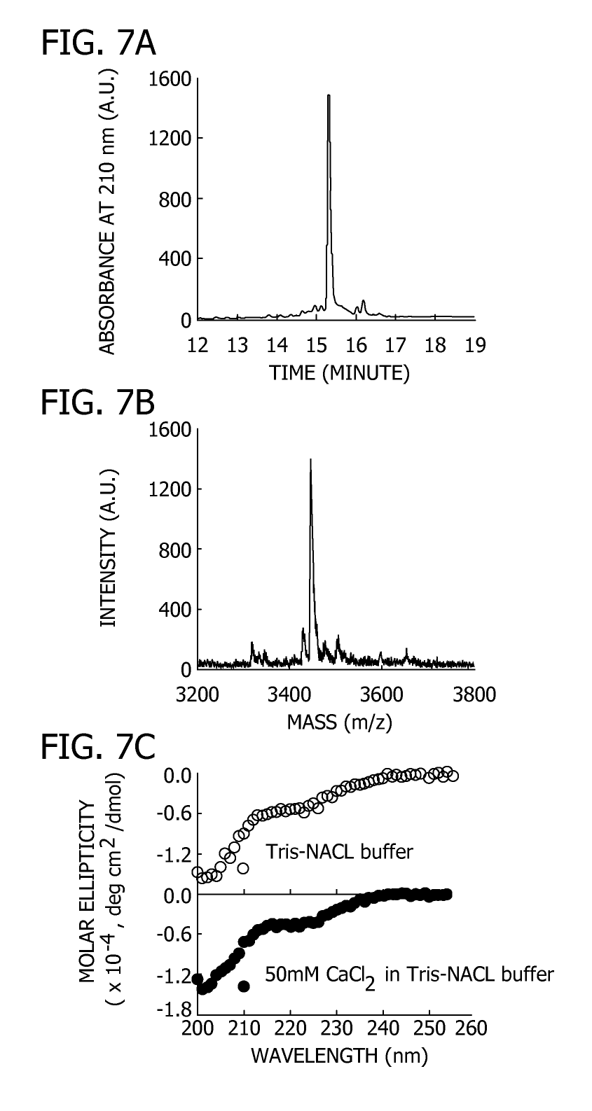

The molecular characteristics of the synthesized modular peptides are shown in FIGS. 7A-7C. The HPLC, MALDI-TOF and CD spectra confirmed that the peptide was successfully synthesized, bearing partial α-helical structure.

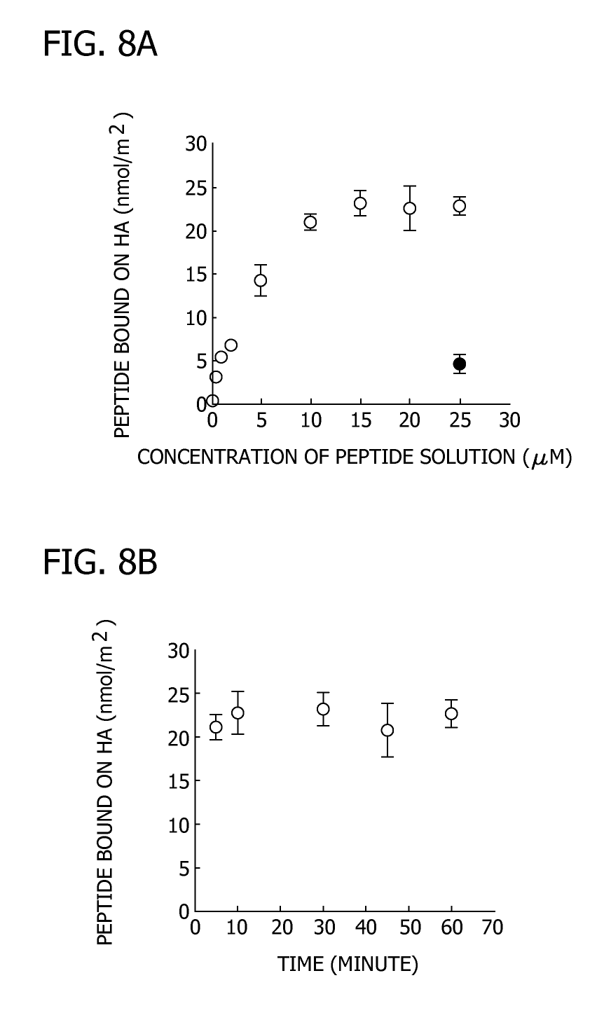

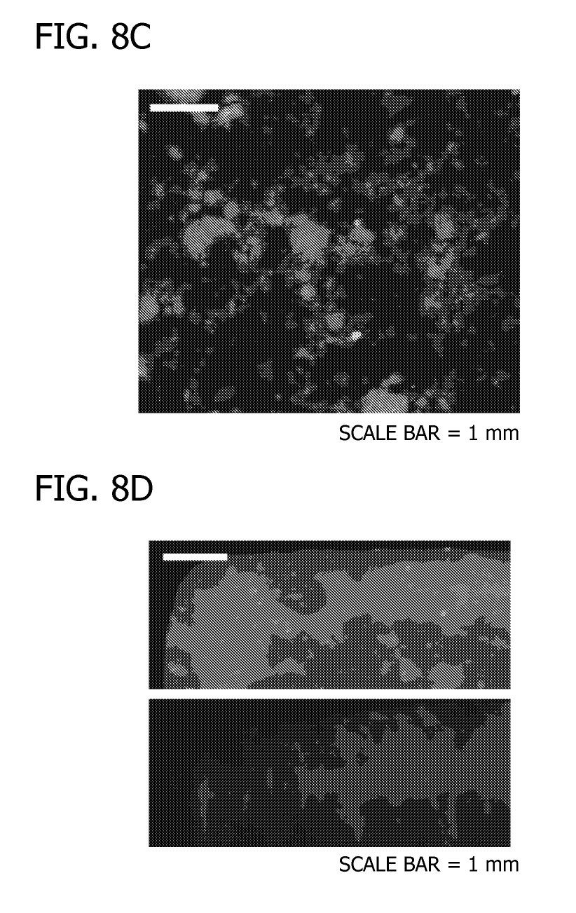

The binding behavior of the peptides (both modular peptide, VEGF-OCN (SEQ ID NO:18), and VEGF-mimic) were analyzed and compared as described above. The amount of bound VEGF-OCN on the HA particle increased with peptide concentration and reached saturation at 15 μM (FIG. 8A). Additionally, it was found that the binding of modular peptide, VEGF-OCN (SEQ ID NO:18) to HA particle was completed within five minutes (see FIGS. 8B and 8C). The amount of VEGF-mimic to HA slab was shown to be much less than that of VEGF-OCN (see FIG. 8D).

### Biological Activity of Modular Peptides

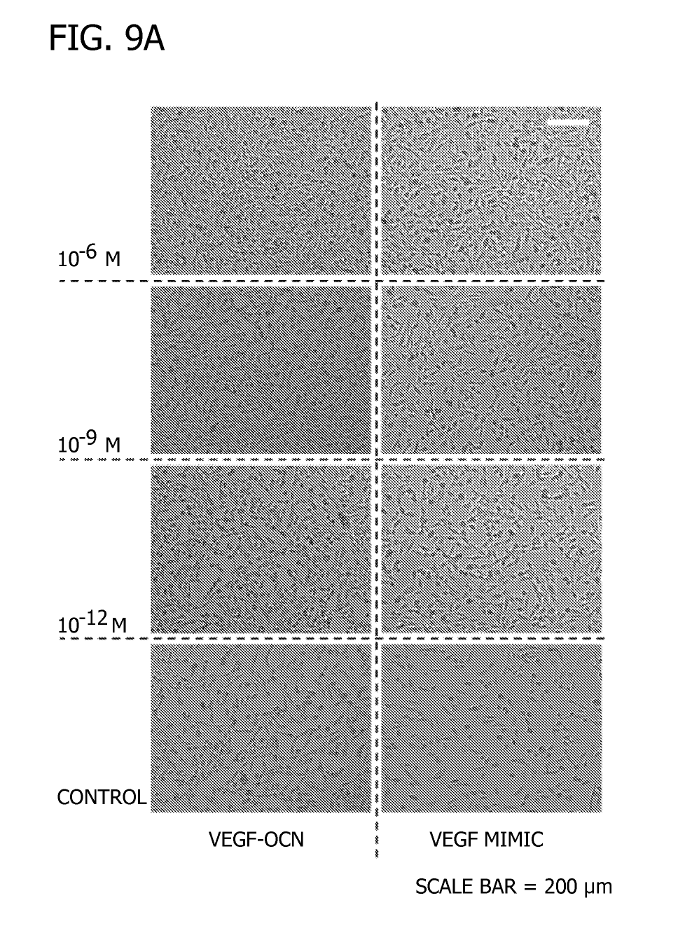

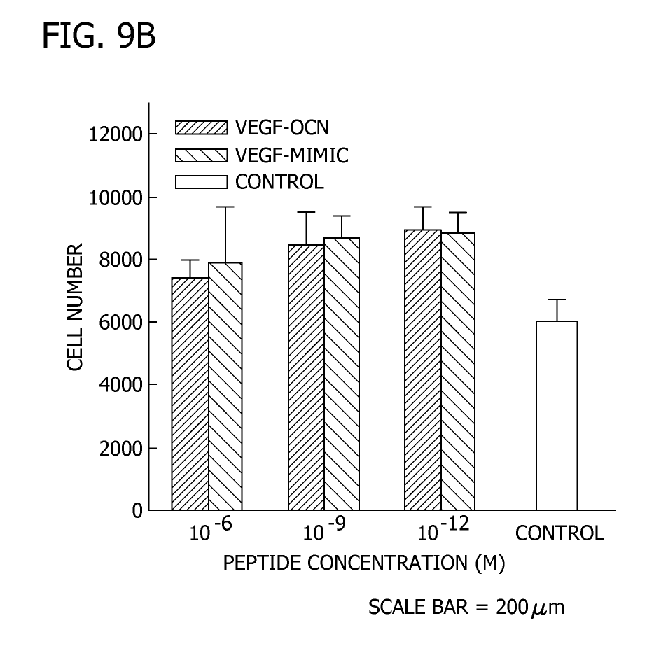

To determine biological activity of VEGF portion for promoting cell proliferation, mouse yolk sac endothelial C166-GFP cells were seeded at a density of 3.12×10³ cells/cm² (1×10³ cells per well) in 96-well plate, allowed to attach for six hours, and then stimulated with either VEGF-OCN or VEGF-mimic After 48 hours of stimulation, optical micrographs were taken using Olympus IX-51 microscope and cell numbers were determined by CYQUANT assay. Results are shown in FIGS. 9A and 9B. Specifically, the results showed that the addition of VEGF-OCN or VEGF-mimic resulted in an increase in cell number to the similar extent when compared to a control, which indicated that the presence of HA-binding portion in VEGF-OCN does not deteriorate the characteristic of VEGF-mimic portion.

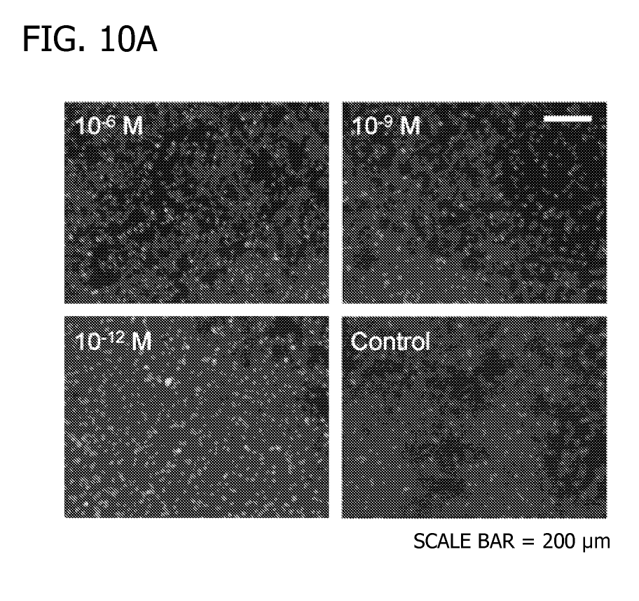

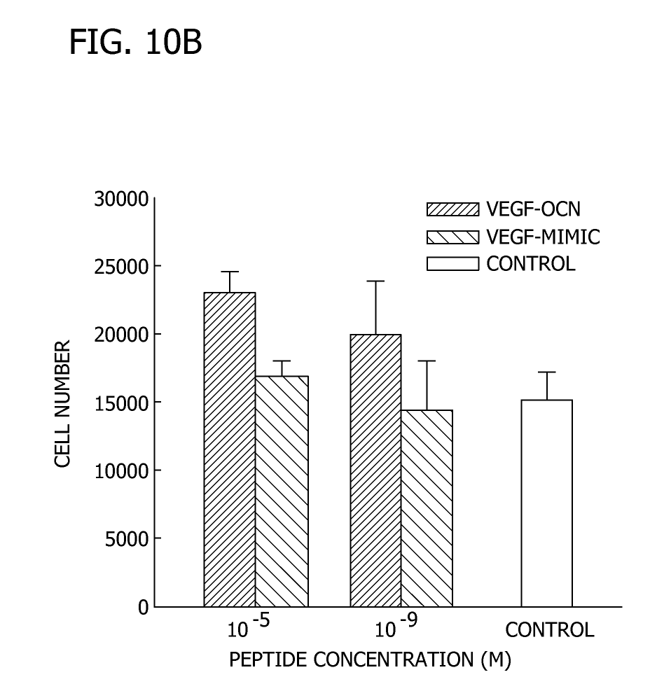

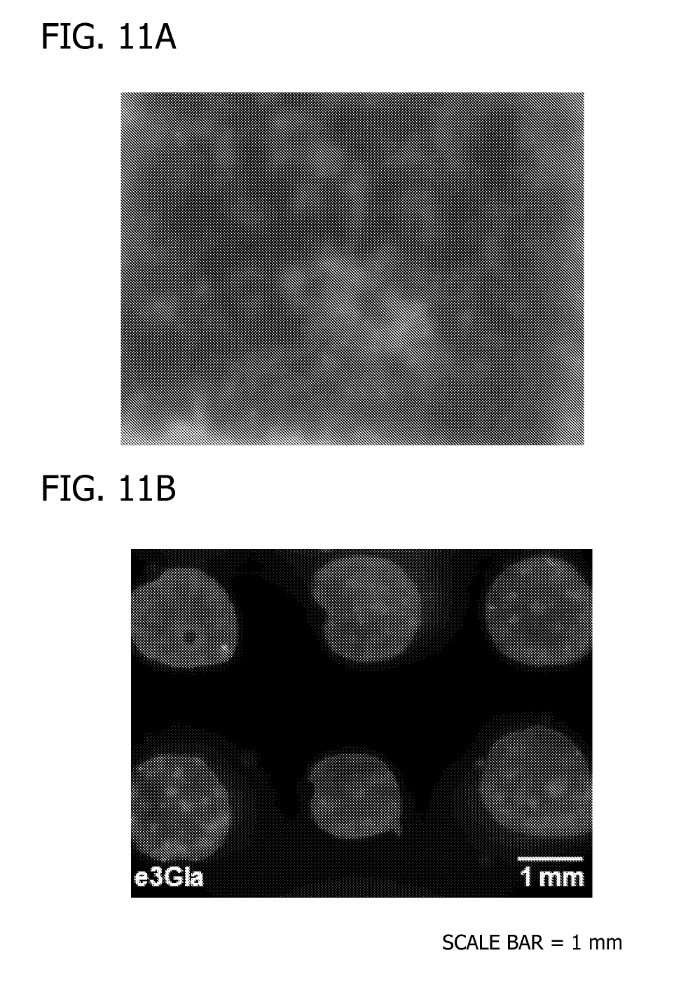

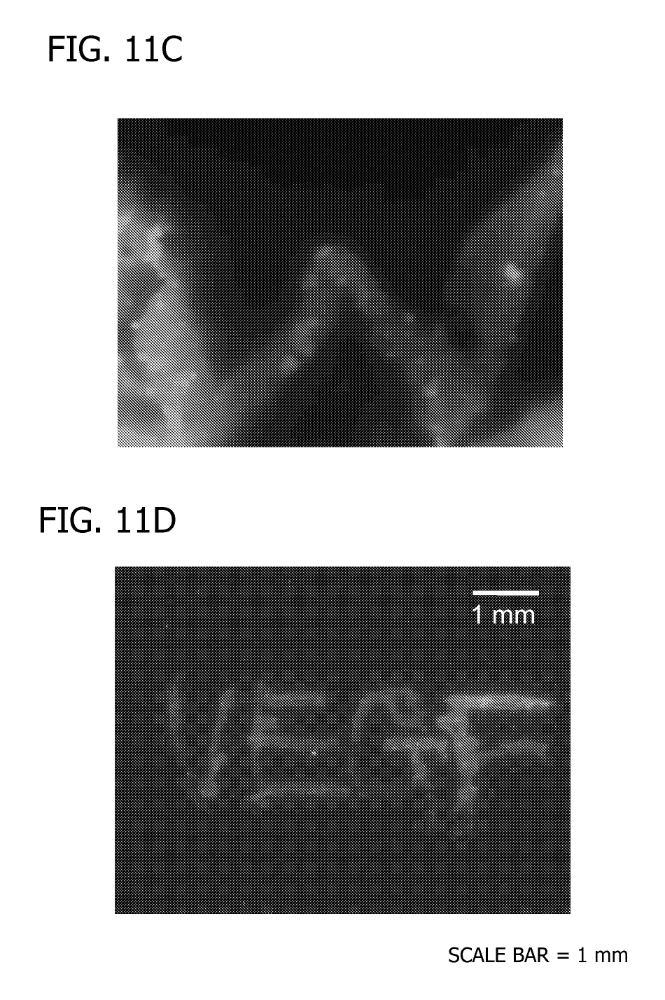

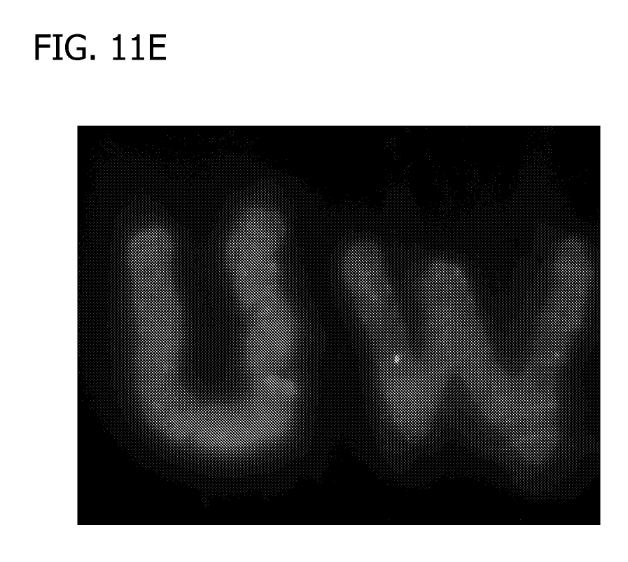

Cells were again seeded at a density of 2×10⁴ cells/cm² (4×10⁴ cells per well) in 24-well plate. Prior to seeding, HA slabs (1 cm×1 cm) were incubated in peptide solution (PBS) for four hours at 37° C. and copiously rinsed with deionized water, and then placed in a well of the 24-well plate. After 1-day culture, cells were treated with 2 μM calcein AM solution, and imaged using Olympus IX-51 microscope. The fluorescence micrographs of C166-GFP cells cultured in the presence of VEGF-OCN or VEGF mimic are shown in FIG. 10A.

Additionally, a cell number count of the C166-GFP cells as cultured in the presence of VEGF-OCN or VEGF mimic was determined For the cell count, cells were seeded at a density of 5×10³ cells/cm² (1×10⁴ cells per well) in a 24-well plate. After 2-day culture, cells were detached from the HA slab and cell number was assessed using CYQUANT assay. As shown in FIG. 10B, there was a significant difference in the increase in the cell number between VEGF-OCN treated and VEGF-mimic treated slabs. This indicated that the VEGF-OCN could bind to the HA slab and promoted cell proliferation. Cell number found on VEGF-mimic treated slab was similar to that of the control, suggesting that VEGF-mimic did not bind to the HA slab, resulting in no stimulation on VEGF-mimic treated HA slab.

### EXAMPLE 3

In this Example, the binding behavior and bioactivity of mBMP to natural bone tissue was analyzed. More specifically, mBMP binding to native bone, either as cadaver bone (allograft model) or in a living bone bioreactor (autograft model) was analyzed.

Specifically, peptides (Table 4) were conjugated with rhodamine to quantify their binding to bone. The rhodamine-labeled peptides were prepared via solid phase peptide synthesis as described in Lee et al. “Modular Peptide Growth Factors For Substrate-Mediated Stem Cell Differentiation,” Angew. Chem. Int. Ed. 2009, 48, 6266-6269.

TABLE 4
Sequences of modular peptide growth factors and natural
template

| Peptide | Amino Acid Sequence |
|---|---|
| OCN template | γEPRRγEVCγEL (SEQ ID NO: 20) |
| BMP2 template | KIPKACCVPTELSAISMLYL (SEQ ID NO: 19) |
| mBMP | KIPKASSVPTELSAISTLYL-AAAA-γEPRRγEVAγEL (SEQ ID NO: 12) |
| mBMP-mut | KIPKASSVPTELSAISTLYL-AAAA-EPRREVAEL (SEQ ID NO: 17) |

The native bone used were harvested from sheep tibia and bovine sternum. Cortical (compact) bone slices were collected from sheep tibia with periosteum removed, and trabecular (cancellous, spongy) bone cores were drilled out from bovine sternum under sterile conditions.

The peptide binding to native bones was tested in three different experiments. In the first experiment, the cortical bone slices were incubated in modular peptide solution (0.5 mL, PBS) with concentrations of 50, 100, 200 and 300 μg/mL. The incubation was continued in a static condition for a period of 0.5, 1, 2 and 3 hours. For another experiment, the trabecular bone cores were placed in the chamber of bone bioreactor where the peptide solution prepared in DMEM (100 μg/mL, 6.5 mL) was continuously circulated through the chamber for a time period of 2, 4, 6, 8 and 10 hours. In the last experiment, mBMP was bound in a spatially controlled manner by dip-coating, spotting or writing with mBMP solution on native bone tissues. Following each experiment, the bones were rinsed with PBS to remove unbound peptide and their fluorescence images were captured using a Typhoon fluorescence scanner (GE healthcare) or a Nikon Eclipse Ti inverted microscope. To quantify the fluorescence intensity, the images were converted into 8-bit using ImageJ to present the intensity level of each pixel in the range of 0 to 255. The mean pixel intensity of a selected region of interest was considered to be proportional to the amount of peptide bound.

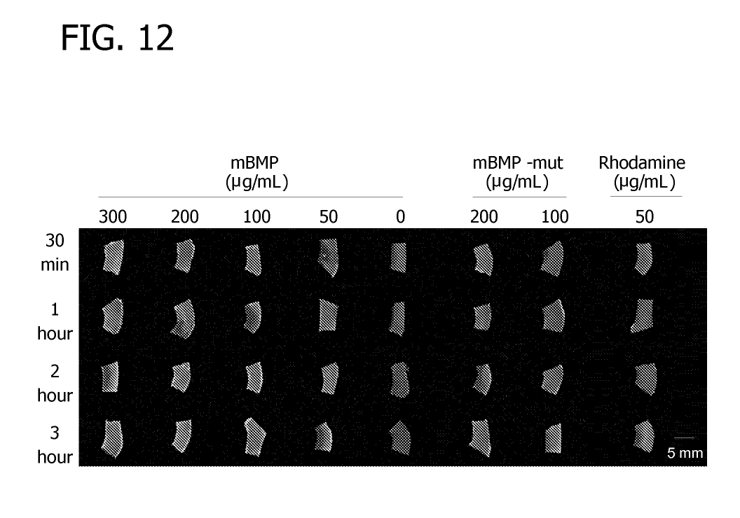

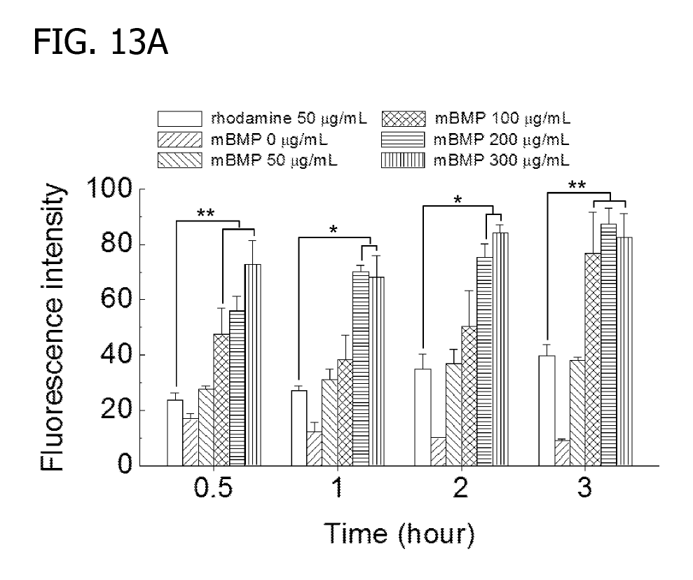

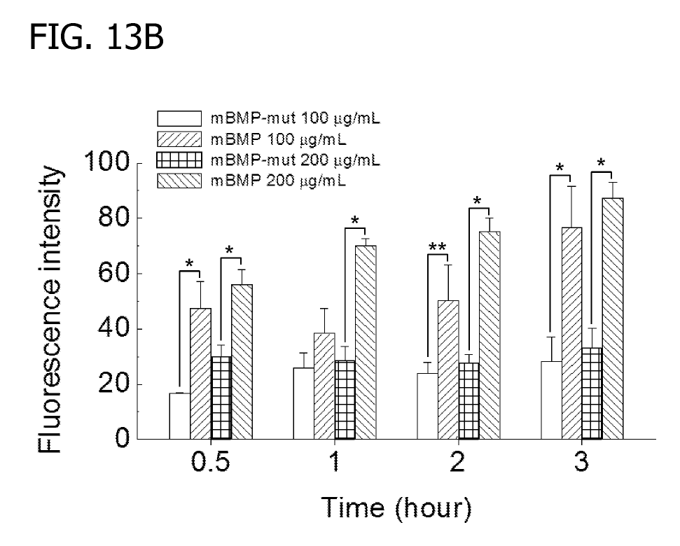

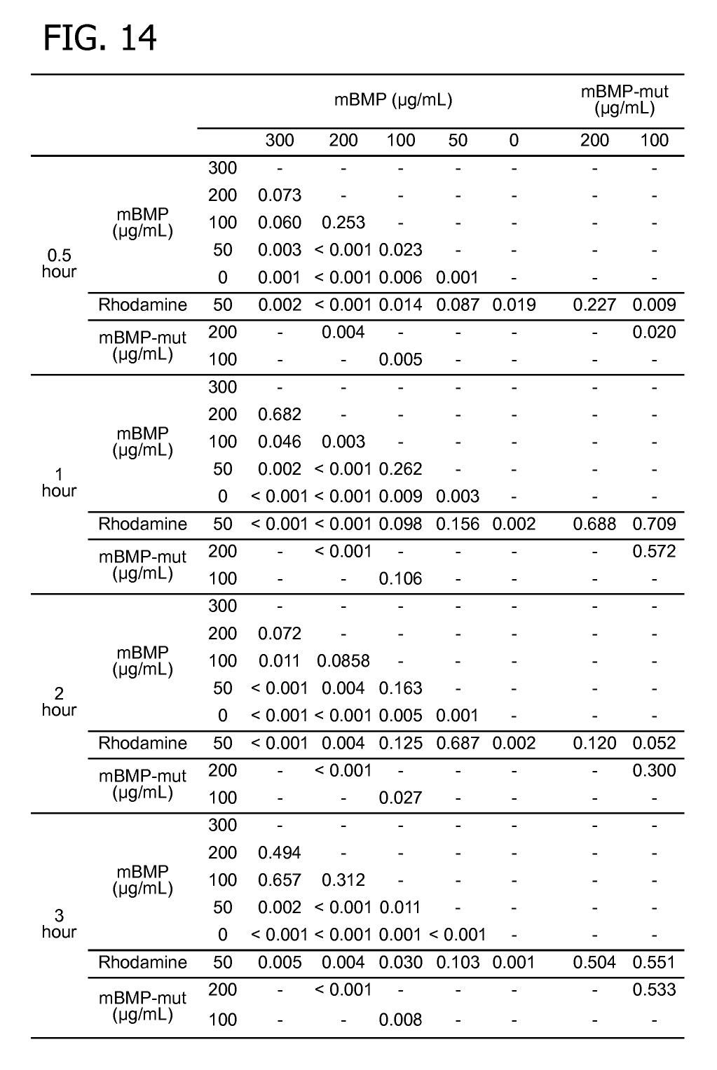

To examine mBMP binding to cortical bone without periosteum (ca. 4 mm×7 mm×1.5 mm) from the shaft of sheep tibia, the bone tissue was incubated in mBMP solution prepared in phosphate buffered saline (PBS). The binding of mBMP was concentration-dependent for each incubation time tested (FIGS. 12 and 13A). The quantity of mBMP binding was proportional to the solution concentration, which was clearly observed through all concentrations at 0.5 hour. However, this dependence was not apparent at higher concentrations at later time points. The concentration of 200 and 300 μg/mL resulted in similar mBMP binding at 1 and 2 hours, and no significant difference was detected among 100, 200 and 300 μg/mL at the 3-hour time point. The detailed statistical analyses are shown in FIG. 14. Interestingly, a statistically significant dependence of mBMP binding on the incubation time was not observed. The insignificant dependence on the incubation time is likely attributed to the rapid binding of mBMP to native bone tissue. The higher fluorescence intensity from mBMP-treated bones when compared with rhodamine-treated group confirmed the specific affinity of mBMP to cadaver bones.

To verify the origin of the high affinity binding of mBMP to bone, the binding of mBMP and a mutated version of mBMP (mBMP-mut in Table 4) were compared. In mBMP-mut, the γE residues were replaced by glutamic acid (E, Glu) (Table 4), which led to a reduced binding affinity to synthetic HAP particles and coatings in previous studies. As expected, the amount of mBMP-mut bound was significantly less than that of mBMP in each experimental condition tested (FIGS. 12 and 13B). The fluorescence intensity level of mBMP-mut treated bones was similar to that of the rhodamine-treated group. Additionally, unlike mBMP binding results, the mBMP-mut binding was independent of incubation time and peptide concentration. The comparison of the binding of mBMP and mBMP-mut confirmed that the high level of bone binding was primarily mediated by γE residues in the HAP-binding motif of mBMP, analogous to the bone binding mechanism of natural OCN protein.

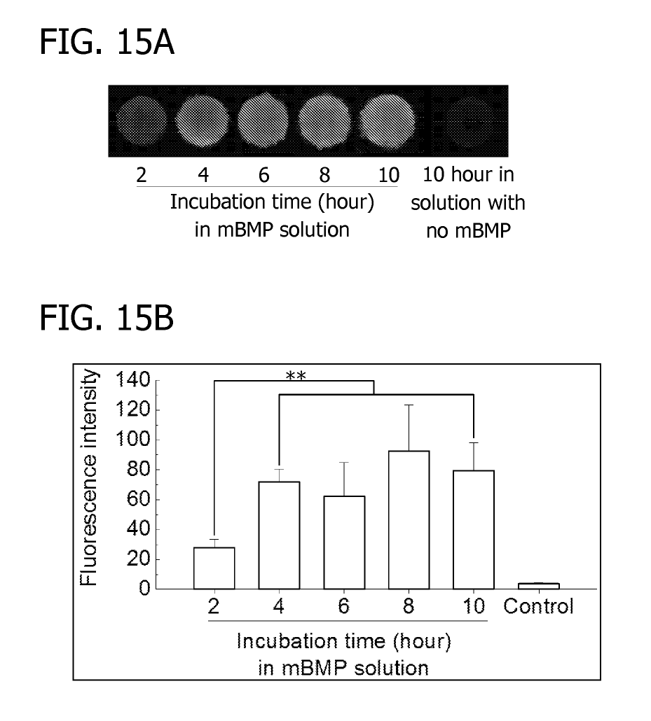

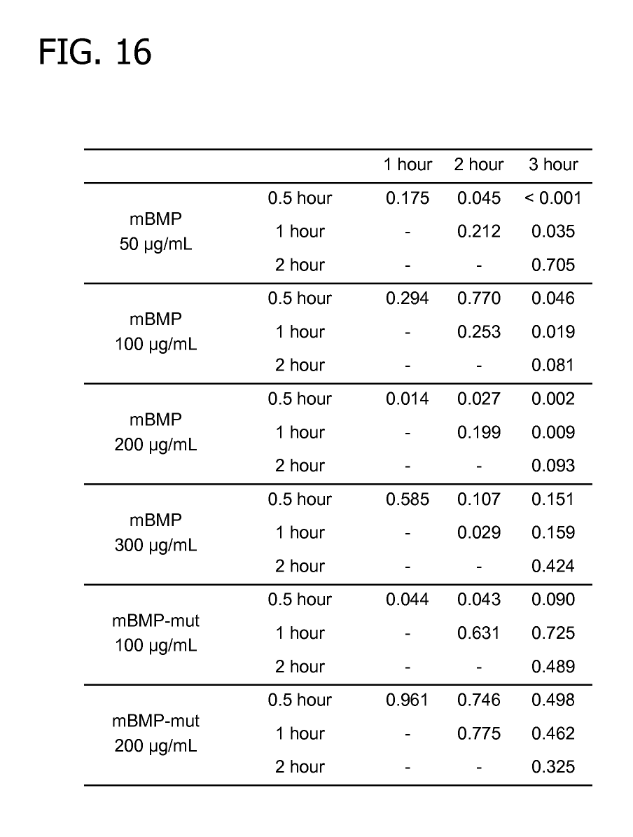

mBMP binding to living trabecular bone cores (1 cm in diameter and 0.5 cm in thickness) harvested from bovine sternum was assessed. For this set of experiments, modular peptide binding occurred in a bone bioreactor where bone cores were kept alive, and a 100 μg/mL mBMP solution in Dulbecco's modified Eagle medium (DMEM) was continuously circulated through the bone perfusion chamber. This bone culture system allows for ex vivo culture of three dimensional trabecular bone explants which included about one million osteocytes, marrow cells and extracellular matrix for several weeks. This experimental platform was used to provide insights into mBMP binding to living bone tissue in a context that may mimic some aspects of direct mBMP injection into bone tissue in vivo. After incubating for various time periods, the binding was measured in terms of fluorescence intensity from rhodamine labeled mBMP. The binding was gradually increased until 4 hours and subsequently reached a plateau (FIGS. 15A-B). Specifically, the binding at longer time points (4, 6, 8 and 10 hours) was significantly higher than binding at 2 hours. However no statistical differences in binding were observed when comparing incubation times longer than 4 hours (FIGS. 15B and 16). This result indicated that the mBMP can incorporate into living trabecular bone.

In summary, mBMP was shown to bind to native bone tissues having different microstructure and porosity (cortical vs. trabecular bone). The quantity and kinetics of mBMP binding was dependent on the concentration of mBMP solution and incubation time. The γE moieties in the HAP-binding, OCN-inspired motif in mBMP were responsible for the high binding affinity to bone tissue. It was also demonstrated that the mBMP could be incorporated into the bone using an ex vivo bone bioreactor. It is noteworthy that localized mBMP binding on cortical bone tissue was also possible using mBMP.

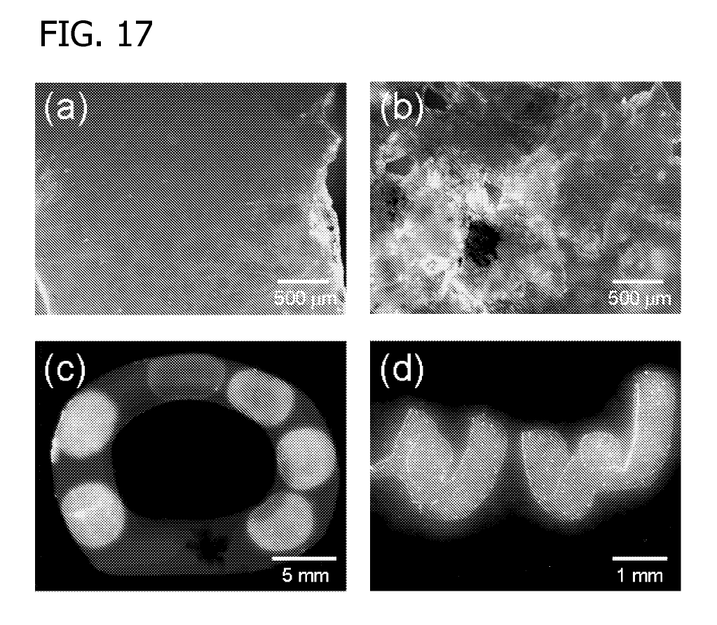

When a bone piece was “dip-coated” in mBMP solution, a significant amount of mBMP was found to bind to the bone surface (FIG. 17A), which indicated that the binding occurred quickly upon contact. Furthermore, the mBMP could be incorporated in a spatially controlled manner by spotting, or direct writing with peptide solution (FIGS. 17B-D). These results suggested that mBMP can be loaded onto the bone with robust spatial control using simple methods that may be easily applied to clinical practice.

In view of the above, it will be seen that the several advantages of the disclosure are achieved and other advantageous results attained. As various changes could be made in the above methods and peptides without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A method of coating native bone with a modular peptide, the method comprising:
exposing native bone to a solution comprising a modular peptide, wherein the native bone is selected from the group consisting of a bone autograft, a bone allograft, and a bone xenograft, wherein the modular peptide comprises a bone-binding portion and a biomolecule-derived portion and wherein the modular peptide is non-covalently bound to the native bone.

2. (canceled)

3. The method of claim 1, wherein the modular peptide is selected from the group consisting of SEQ ID NO:12, SEQ ID NO:13, SEQ ID NO: 14, SEQ ID NO:15, SEQ ID NO:16, SEQ ID NO:17, and SEQ ID NO:18.

4. The method of claim 1, wherein the bone-binding portion comprises an amino acid sequence selected from the group consisting of SEQ ID NO:1, SEQ ID NO:2, SEQ ID NO:3, SEQ ID NO:4, SEQ ID NO:5, and SEQ ID NO:6.

5. The method of claim 1, wherein the biomolecule-derived portion initiates at least one of osteoconduction, osteogenesis, angiogenesis, and osteogenic differentiation.

6. The method of claim 1, wherein the biomolecule-derived portion comprises an amino acid sequence selected from the group consisting of SEQ ID NO:8, SEQ ID NO:9, SEQ ID NO:10, and SEQ ID NO:11.

7. The method of claim 1, wherein the modular peptide further comprises a spacer portion.

8. The method of claim 7, wherein the spacer portion is an amino acid sequence capable of forming an α-helix.

9. The method of claim 7, wherein the spacer portion is SEQ ID NO:7.

10. The method of claim 1, wherein the solution is selected from the group consisting of PIPES buffer solution, Tris buffer solution, saline solution.

11. The method of claim 1, wherein exposing native bone to a solution comprises at least one of dip coating, painting, stamping, spotting, and brushing.

12. The method of claim 1, wherein exposing native bone to a solution comprising a modular peptide comprises exposing native bone to the solution under constant agitation.

13-20. (canceled)
